(19) United States
(12) Patent Application Publication
(10) Pub. No.: US 2002/0132276 A1
(43) Pub. Date: Sep. 19, 2002

(54) FACS method for detecton of GPIIb/IIIa inhibitor dependent activators in plasma samples

(76) Inventors: Ira B. Dicker, Wilmington, DE (US); Susan M. Spitz, Wilmington, DE (US); Beth E. Thomas, Newark, DE (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/491,796

(22) Filed: Jan. 26, 2000

Related U.S. Application Data
(60) Provisional application No. 60/117,251, filed on Jan. 26, 1999.

(51) Int. Cl. G01N 33/53

(52) U.S. Cl. 435/7.24

(57) ABSTRACT

This invention relates to assays useful for the detection in a patient bodily fluid sample of drug-dependent substances that bind to integrins, or integrin-associated proteins or complexes thereof in the presence of an integrin antagonist/agonist. This invention also relates to assays useful for the detection in a patient body fluid sample of drug-dependent cell activating substances (DDPASs) whose action on the cells depends on the binding of a integrin antagonist/agonist. This invention also relates to the use of platelet activation markers to detect integrin antagonist/agonist dependent DDPASs.

### FIELD OF THE INVENTION

This invention relates to assays useful for the detection in a patient bodily fluid sample of drug-dependent substances that bind to integrins, or integrin-associated proteins or complexes thereof in the presence of an integrin antagonist/agonist. This invention also relates to assays useful for the detection in a patient body fluid sample of drug-dependent platelet activating substances (DDPASs) whose action on the cells depends on the binding of an integrin antagonist/agonist. This invention also relates to the use of platelet activation markers to detect integrin antagonist/agonist dependent DDPASs.

### BACKGROUND OF THE INVENTION

Thromboembolic diseases, including stable and unstable angina pectoris, myocardial infarction, stroke and lung embolism, are the major cause of disability and mortality in most developed countries. Recently, therapeutic strategies aimed at interfering with the binding of ligands to the GPIIb/IIIa integrin have been explored to treat these patient groups. Platelet GPIIb/IIIa is the main platelet receptor for fibrinogen and other adhesive glycoproteins, including fibronectin, vitronectin and von Willebrand factor. Interference of ligand binding with this receptor has been proven beneficial in animal models of thromboembolic disease (Coller, B. S. GPIIb/IIIa Antagonists: Pathophysiologic and Therapeutic Insights From Studies of C7E3 FAB. Thromb. Haemost. 78: 1, 730-735, 1997), and in limited studies involving human subjects (White, H. D. Unmet Therapeutic Needs in the Management of Acute Ixchemia. Am. J. Cardiol. 80: 4A, 2B-10B, 1997; Tcheng, J. E. Glycoprotein IIb/IIIa Receptor Inhibitors: Putting EPIC, IMPACT II, RESTORE, and EPILOG Trials Into Perspective. Am. J. Cardiol. 78: 3A, 35-40, 1996).

A number of cell surface receptor proteins, referred to as integrins or adhesion protein receptors, have been identified which bind to extracellular matrix ligands or other cell adhesion protein ligands thereby mediating cell-cell and cell-matrix adhesion processes. The integrins are encoded by genes belonging to a gene superfamily and are typically composed of heterodimeric transmembrane proteins containing α- and β-subunits. Integrin subfamilies contain a common β-subunit combined with different α-subunits to form adhesion protein receptors with different specificities. In addition to GPIIb/IIIa, a number of other integrin cell surface receptors have been identified. For example, members of the β1 subfamily, α4β1 and α5β1, have been implicated in various inflammatory processes, including rheumatoid arthritis, allergy, asthma and autoimmune disorders.

The integrin GPIIb/IIIa, also referred to as the platelet fibrinogen receptor, is the membrane protein mediating platelet aggregation. GPIIb/IIIa in activated platelets is known to bind four soluble RGD containing adhesive proteins, namely fibrinogen, von Willebrand factor, fibronectin, and vitronectin. The term “RGD” refers to the amino acid sequence Arg—Gly—Asp. The binding of fibrinogen and von Willebrand factor to GPIIb/IIIa causes platelets to aggregate. The binding of fibrinogen is mediated in part by the RGD recognition sequence which is common to the adhesive proteins that bind GPIIb/IIIa. RGD-peptidomimetic GPIIb/IIIa antagonist compounds are known to block fibrinogen binding and prevent platelet aggregation and the formation of platelet thrombi. GPIIb/IIIa antagonists represent an important new approach for anti-platelet therapy for the treatment of thromboembolic disorders.

Approximately 1% of individuals receiving certain GPIIb/IIIa antagonists develops life-threatening thrombocytopenia. The principal cause of these thrombocytopenias is thought to be immune mediated, due to the presence of drug-dependent anti-platelet antibodies (Berkowitz, S. D., Harrington, R. A., Rund, M. M., and Tcheng, J. E. Acute Profound Thrombocytopenia After C7E3 FAB (abciximab) Therapy. Circulation 95:809-813, 1997). However, such drug-dependent anti-platelet antibodies have not been found in all patients undergoing GPIIb/IIIa inhibitor treatment, leading to speculation that there may be other causes for GPIIb/IIIa-inhibitor-dependent thrombocytopenia.

The general phenomenon of drug-dependent thrombocytopenia/thromboembolic complications is well known. Clinically important examples are heparin-induced thrombocytopenia (HIT) (Amiral, J., Bridley, F., Wolf, M., et al., Antibodies to macromolecular platelet factor IV-heparin complexes in heparin-induced thrombocytopenia: A study of 44 cases. Thromb. Haemost. 1995, 73:21-28; Ansell, J., Deykin, D., Heparin-induced thrombocytopenia and recurrent thromboembolism. Am. J. Hematol. 1980, 8:325-332), and heparin-induced thrombotic thrombocytopenia (HITT), though many other drugs have been implicated (Kelton, J. G., Sheridan, D. P., Santosi A. V., et al. Heparin-induced thrombocytopenia: Laboratory studies. Blood, 1988, 72:925-930; Chong, B., Berndt, M. Heparin induced thrombocytopenia. Blut 1989, 58:53-57; Curtis, B. R., McFarland, J. G., Wu, G -G., Visentin, G. P., and Aster, R. H., Antibodies in sulfonamide-induced immune thrombocytopenia recognize calcium-dependent epitopes on the glycoprotein IIb/IIIa complex. Blood, 1994 84:176-183). HIT and HITT are thought to be of immune origin involving binding to the platelet of drug-dependent anti-platelet antibodies induced by the formation of heparin/platelet Factor IV/antibody complexes (Karpatikin, S., Drug-induced thrombocytopenia. 1971, Amer. J. Medical Sciences, 262:68-78). Platelet clearance is thought to be mediated by the reticuloendothelial system (RES). In some cases such drug/antibody complexes are reported to activate platelets, leading directly to platelet secretion and aggregation (Amiral, J., wolf, M., Fisher, A. M., Boyer-Neumann, C., Vissac, A. M., and Meyer, D. Pathogenicity of IgA and/or IgM antibodies to heparin-platelet Factor IV complexes in patients with heparin-induced thrombocytopenia. British J. of Haem. 1996, 92:954-959). However, antibodies have not been detected in all cases, thus there may be non-immune mechanisms for heparin and other drug-dependent thrombocytopenias.

Cases of thrombocytopenia of unknown origin are referred to as idiopathic thrombocytopenic purpura (ITP). In most patients this disorder is thought to be caused by autoantibodies against platelet membrane glycoproteins (Gonzalez-Conejero, R., Rivera, J., Rosillo, M. C., Lozano, M. L., and Garcia, V. V., Comparative study of three methods to detect free plasma antiplatelet antibodies. Acta Haematol., 96:135-139, 1996; Stockelber, D., Hou, M., Jacobson, S., Kutti, J., Wadenvik, H., Detection of platelet antibodies in chronic idiopathic thrombocytopenic purpura (ITP). A comparative study using flow cytometry, a whole platelet ELISA, and an antigen capture ELISA. Eur. J. Haematol., 56:72-77, 1996) and possibly glycolipids (Arnout, J. The pathogensis of the antiphospholipid syndrom: A hypothesis based on parallelisms with heparin-induced thrombocytopenia. Thrombosis and Haemostasis, 75:536-541, 1996; Cuadrado, M. J., Mujic, F., Munoz, E., Khamashta, M. A., Hughes, G. R. V., Thrombocytopenia in the antiphospholipid syndrom. Annals of the Rheumatic Diseases, 56:194-196, 1997), with removal of IgG-sensitized platelets by the RES. However, autoantibodies are not detected in all cases thus, there may be non-immune mechanisms for ITP.

Activators of the basic platelet reaction are capable of causing thrombocytopenia, as is observed by the participation of thrombin in disseminated intravascular coagulation (DIC, Minna, J. D., Robboy, S. J., Colman, R. W. DIC in Man. Springfield, Ill., Charles C. Thomas, 1974). Another process of platelet activation resulting in subsequent thrombocytopenia is thrombotic thrombocytopenic purpura (TTP). Though the pathogenesis is uncertain, there is evidence for a circulating “toxic” factor which activates platelets, and leads to their removal from the circulation (Murphy, W. G., Moore, J. C., Kelton, J. G. Calcium-dependent cysteine protease activity in the sera of patients with thrombotic thrombocytopenic purpora, Blood 70:1683, 1987).

A possible mechanism of action is that the binding of GPIIb/IIIa antagonists to GPIIb/IIIa on the platelet surface renders the platelet more sensitive to the action of platelet activators, for example, the extent of platelet activation will be greater in the presence versus the absence of the GPIIb/IIIa antagonist. An activating function for the binding of GPIIb/IIIa antagonists has been noted in that some antagonists may act as partial agonists of integrin function (GPIIb/IIIa affinity state and aggregation, Du X. P, Plow, E. F, Frelinger, A. L. 3d, O'Toole, T. E, Loftus, J. C., Ginsberg, M. H. Ligands “activate” integrin alpha IIb beta 3 (platelet GPIIb/IIIa). Cell, 1991, 65(3):409-416) and fibrinogen binding (Peter, K,, Schwarz, M., Ylanne, J., Kohler, B., Moser, M., Nordt, T., Salbach, P., Kubler, W., Bode, C. Induction of fibrinogen binding and platelet aggregation as a potential intrinsic property of various glycoprotein IIb/IIIa (IIbbeta3) inhibitors. Blood, 92(9):3240-9, 1998). Such activators may include, but are not limited to ADP, platelet activating antibodies, drug-dependent platelet activating antibodies, and other activators of the basic platelet reaction (Hemostasis and Thrombosis: Principles and Clinical Practice, third Edition, Coleman, R. W., Hirsh, J., Marder, V. J., Salzman, E. W., and Holmsen, J. B., eds., Chapter 24: Platelet secretion and energy metabolism. Lippincott Company, Philadelphia, Pa., 1994) including thrombin, epinephrine, collagen, arachidonate and the thrombin receptor activating peptide, TRAP (Brass, L. F., et al., The human platelet thrombin receptor. Turning it on and turning it off. Ann. N.Y. Acad. Sci., 714:1-12, 1994).

The complications associated with the use of GPIIb/IIIa antagonist/agonists may severely limit their use, and integrin antagonist/agonists in general, because patients may develop a thrombocytopenic/thromboembolic episode mediated by either drug dependent platelet activating substances (DDPASs), and/or DDABs, and/or other drug-dependent mechanisms.

GPIIb/IIIa DDPASs are defined here as substances that

(a) bind to and activate platelets in the presence of a GPIIb/IIIa antagonist but do not bind to or activate platelets in the absence of a GPIIb/IIIa antagonist, or

(b) which bind to platelets in the absence of a GPIIb/IIIa antagonist, but whose ability to induce platelet activation is potentiated by GPIIb/IIIa antagonists.

GPIIb/IIIa DDPASs may bind, for example, to stable neoepitopes in GPIIb/IIIa and/or GPIIb/IIIa-associated proteins or complexes, which are mediated or induced by the binding of the GPIIb/IIIa antagonist to GPIIb/IIIa. The DDPASs may also bind to unstable neoepitopes requiring the constant presence of GPIIb/IIIa and/or GPIIb/IIIa-associated proteins or complexes, and the antagonist, or to structural entities consisting of GPIIb/IIIa and/or GPIIb/IIIa-associated proteins or complexes, and the antagonist/agonist itself. DDPASs may be DDABs (see commonly-owned pending U.S. patent application Ser. No. 09/237061, filed Jan. 26, 1999, the contents of which are herein incorporated by reference), or DDPASs may not be DDABs.

It follows from the foregoing considerations that a sensitive and specific assay that can detect such GPIIb/IIIa directed DDPASs and DDABs may be beneficial in identifying patients with such DDPASs and DDABs which are present prior to treatment with the GPIIb/IIIa antagonist, and/or DDPASs or DDABs which develop and increase in titer following administration of the GPIIb/IIIa antagonist. Patients with pre-existing or developing DDPAS or DDAB titer may have a greater risk of undergoing thrombocytopenic/thromboembolic episodes following administration of the GPIIb/IIIa antagonist. Patients that are determined to have pre-existing DDPASs or DDABs may either be excluded from therapy with GPIIb/IIIa antagonists, or may be treated with a compound which is less prone to potentiate the binding/activation by DDPASs. Alternatively, if a DDPAS or DDAB titer should develop, the therapy can be stopped prior to the onset of a clinically significant thrombocytopenic/thromboembolic episode. Patients with pre-existing DDPASs or DDABs may be at risk of developing a thrombocytopenic/thromboembolic episode upon treatment with GPIIb/IIIa antagonist.

Low titers of pre-existing DDPASs or DDABs may be present in a relatively large percentage of the general population. It follows that procedures aimed at identifying patients in the DDPASs-positive population that are at increased risk for thrombocytopenia/thromboembolic complications will facilitate the exclusion of this “high risk” population from therapy with a specific GPIIb/IIIa antagonist, treatment with chemically distinct GPIIb/IIIa antagonists, or identify patients in need of extensive monitoring during treatment.

In patients with developing or increasing DDPAS or DDAB titer, the identification of such an increase at the earliest time point is necessary to exclude, terminate and/or change therapeutic modalities with a specific GPIIb/IIIa antagonist prior to the development of a clinically significant thrombocytopenic/thromboembolic episode. A number of procedures aimed at recovering platelet-associated antibodies are known in the art. They require isolation of platelets from whole blood and treatment with low or high pH, or protein denaturants. These procedures can only be performed in specialized laboratories on freshly prepared biological specimens. In addition, false-negative results are to be expected due to inherent instabilities of specific antibodies, excluding a reliable functional analysis of the resulting platelet eluate. Ethylenediaminetetraacetic acid (EDTA) treatment of isolated platelets has been reported to dissociate the GPIIb/IIIa complex, and reduced binding of conformationally sensitive murine antibodies to GPIIb/IIIa has been observed. The use of EDTA treatment in whole blood using DDPASs, human autoantibodies to GPIIb/IIIa or DDABs directed to GPIIb/IIIa has not been reported.

The utility of assays aimed at detecting DDPASs and DDABs can be increased if reliable DDPAS and DDAB standards are available. The standard should be reactive with the same secondary antibody detection system as the human DDAB and thus allow for a calibration of the experimental results. The method and composition of such a standard has not been taught in the art.

There remains the need for sensitive, specific and easy-to-use assays to be used in conjunction with integrin antagonist/agonist treatment, such assays being capable of detection of low levels of integrin antagonist/agonist DDPASs which may be present in an individual prior to the administration of an integrin antagonist/agonist and/or for the detection of developing DDPASs following treatment with the integrin antagonist/agonist. The present invention provides such assays for the detection of integrin antagonist/agonist DDPASs.

There is a continuing need to increase the sensitivity, specificity, and ease of use of methods to detect DDPASs to integrins. The present invention provides such procedures for the detection of integrin-directed platelet activating substances.

P-selectin, also known as CD62, GMP-140, or PADGEM, is a member of the selectin family of adhesion receptors that regulates leukocyte trafficking (Lawrence, M. B. and T. A. Springer, Cell, 65:859 (1991); Johnston, G. I. et al., Cell, 56: 1033-1044 (1989); U.S. Pat. No. 5,378,464). P-selectin is an intergral membrane glycoprotein found in the α-granules of unactivated platelets and in the Weibel-Palade bodies of endothelial cells (Peerschke, E. I. B., Am. J. Clin. Pathol., 98: 455 (1992); McEver, R. P., 1993, Leukocyte Interactions Mediated By P-selectin, in: Structure, Function and Regulation of Molecules Involved in Leukocyte Adhesion, Lipsky, P. E., et al., Eds., Springer-Verlag, N.Y., pp. 135-150). P-selectin is a sensitive marker for platelet activation. Activation of platelets by antagonists results in the translocation of P-selectin from the secretory granules to the cell surface (Stenberg, P. E. et al., 1985, “A platelet alpha granule membrane protein (GMP-140) is expressed on the plasma membrane after activation”, J. Cell Biol., 101:880-886 (1985)).

Various methods have been reported for the detection of p-selectin on the platelet surface. These include flow cytometry: Shattil, S. J., Cunningham, M. and Hoxie, J. A. Detection of activated platelets in whole blood using activation-dependent monoclonal antibodies and flow cytometry. Blood 70, 307-315, 1987; radioimmunoassay: George, J. N., Pickett, E. B., Saucerman, S., Mcever, R. P., Junicki, T. J., Kieffer, N. and Newman, P. J. Platelet surface glycoproteins: Studies on resting and activated platelet membrane microparticles in normal subjects and observations in patients during adult respiratory distress syndrome and cardiac surgery. J. Clin. Invest. 78:340-348, 1986; Immunocytochemistry: Stenberg, P. E., McEver, R. P., Shuman, M. A., Jacques, Y. V. and Bainton, D. F. A platelet alpha-granule membrane protein (GMP-140) is expressed on the plasma membrane after activation. J. Cell Giol. 101:880-886, 1985; the platelet/neutrophil resetting assay: Dembinska-Kiec, A., Zmunda, A., Wenhrynowicz, O., Stachura, J., Peskar, B. A. and Gryglewski, R. J. P-selectin-mediated adherence of platelets to neutrophils is regulated by prostanoids and nitric oxide. Int. J. Tissue Reactions 15:55-64, 1993; Fluoroescence-conjugated immunobinding assay: Wen, D., Nguyen, T. T., Plumhoff, E. A., Pineda, A. A., Bowie, E. J. W. and Kottke, B. A. A fluorescence-conjugated immunobinding assay for the detection of P-selectin on platelets. J. Lab. Clin. Med. 124:447-454, 1994; and an ELISA assay: Whiss, P. A., Andersson, R. G. G. and Srinivas, U. Modulation of P-selectin expression on isolated human platelets by a NO donor assessed by a novel ELISA application. J. Imm. Methods, 200:135-143, 1997. Kottke, B. A., et al. (Watson Clinic), (WO 96/12956) describe a flow cytometric assay for the detection of p-selectin on the platelet (Fluorescence-conjugated immunoassay for platelet expression in a sample of whole blood in vitro). Dalesandro, M. R. and Frederick, B., (WO 98/21591) describe a flow cytometic assay for the detection of the effects of “anti-platelet” agents on the level of p-selectin on the platelet surface. None of the above-mentioned methods has been reported to measure a drug-dependent increase in the exposure of p-selectin on the platelet surface in general, or a GPIIb/IIIa-antagonist dependent increase in the exposure of platelet p-selectin, in particular.

Detection of activated platelets mediated by a drug dependency is represented by assays for the measurement of heparin-dependent antibodies causative for HIT which utilize markers of platelet activation as their endpoint. ¹⁴C-serotonin release is also commonly used for the detection of heparin-dependent antibodies. For example Favaloro et al.(Favaloro, E. J, Bernal-Hoyos, E, Exner, T, Koutts, J., Heparin-induced thrombocytopenia: Laboratory investigation and confirmation of diagnosis, Pathology 1992, 24(3):177-183) report on the usefulness of the ¹⁴C-serotonin release assay for the laboratory confirmation of the clinical diagnosis of heparin-induced thrombocytopenia syndrome (HITS).

Tomer, A. (Department of Medicine, Emory University, Atlanta) reports a functional flow cytometric assay (FCA) for the diagnosis of heparin-induced thrombocytopenia (HIT) (A sensitive and specific functional flow cytometric assay for the diagnosis of heparin-induced thrombocytopenia, Br. J. Haematol. 1997, 98(3):648-656). This method uses flow cytometry to measure the heparin-induced binding of fluorescently labeled annexin V to platelets in the presence of patient sera containing platelet-activating, heparin-dependent antibodies. Platelet dense granule release other than ¹⁴C serotonin has also been utilized for the detection of heparin-dependent antibodies. For example, Stewart et al. (Stewart, M. W., Etches, W. S., Boshov, L. K. and Gordon, P. A., Wai, S. W., Heparin-induced thrombocytopenia: An improved method of detection based on lumi-aggregometry, Br. J. Haematol. 1995, 91:173-177), report on the use of lumi-aggregometry to detect the release of ATP as an endpoint of HIT antibody-mediated platelet activation. The use of either the ¹⁴C-serotonin release or ATP release from dense granules of the platelet or the use of annexin V binding methods depends on the presence of sufficiently activating antibodies or other platelet activating substances in the patient sera. Only substantially activating species are capable of inducing such dense granule release or eliciting the change in the platelet surface that could support the binding of the protein label, annexin V. Therefore, there is a need for more sensitive and specific detection methods for drug-dependent platelet activating substances, which is satisfied by the present invention.

### SUMMARY OF THE INVENTION

This invention provides treatment methods and procedures to identify patients at risk for integrin antagonist/agonist mediated disease states. The present invention provides assays and methods useful for the detection, in a patient bodily fluid sample, of drug-dependent substances that bind to and/or activate cells in the presence of an integrin antagonist/agonist. The present invention provides sensitive, specific and easy-to-use assays that may be used in conjunction with integrin antagonist/agonist treatment. These assays are capable of detection of low levels of integrin antagonist/agonist-dependent substances that bind to and/or activate cells which may be present in an individual prior to the administration of an integrin antagonist/agonist, and/or for the detection of developing integrin-antagonist/agonist-dependent substances following treatment with the integrin antagonist/agonist.

This invention relates to the use of platelet activation markers to detect platelet activation in the presence of integrin antagonists/agonists. The present invention provides a flow cytometric method using whole platelets and certain GPIIb/IIIa antagonists and detects the presence, on the platelet surface, of the platelet activation protein p-selectin, herein referred to as CD62. The GPIIb/IIIa flow cytometric assay of the present invention detects pre-existing GPIIb/IIIa platelet activating substances (i.e., platelet activating substances which are pre-existing in the patient prior to the patient being administered the GPIIb/IIIa antagonist). The GPIIb/IIIa platelet activating substance flow cytometric assay of the present invention also detects GPIIb/IIIa platelet activating substances for which an increased titer of the platelet-activating substance develops following the GPIIb/IIIa antagonist being administered to the patient, the action of such GPIIb/IIIa platelet activating substances being potentiated by the presence of the GPIIb/IIIa antagonists. The present assays and methods may be used to identify individuals having GPIIb/IIIa antagonist-induced platelet activating substances and may be used to exclude, terminate, and/or change therapeutic modalities with GPIIb/IIIa antagonists prior to the onset of thrombocytopenia/thromboembolic complications.

The present assays may be used to identify patients at risk of developing GPIIb/IIIa antagonist-induced thrombocytopenia or thromboembolic complications and/or to identify patients who are not at risk of developing GPIIb/IIIa antagonist-induced thrombocytopenia or thromboembolic complications.

### BRIEF DESCRIPTION OF THE DRAWINGS

FIG. 1: Use of the GPIIb/IIIa DDPASFCA to detect platelet activating drug-dependent antibodies (PADDABs) (Example 5)

PRP at three dilutions was incubated with increasing concentrations of murine JK094 antibody in the presence and absence of 1000 nM compound A as described in Example 1. Platelet-associated P-selectin expression was measured as described in Example 1.

FIG. 2: DDPASFCA of thrombocytopenic patient #304 (Example 8)

The DDPASFCA analysis of patient #304 at 22 days and 17 months post Compound A administration versus positive control DPC38 and negative control DPC3. Note the assay indicates that this individual was DDPASFCA positive at the earliest time point evaluated, suggesting the utility of this assay to monitor patients before, during and after GPIIb/IIIa antagonist treatment to identify patients with DDPASs. In a prospective study, a patient with such pre-existing DDPASs could be excluded from the study, possibly preventing the clinically significant thrombocytopenic/thromboembolic episode.

FIG. 3: Specific GPIIb/IIIa antagonist-induced distribution of thrombocytopenic patient 099016 DDPAS onto platelets and recovery by EDTA elution (Example 9)

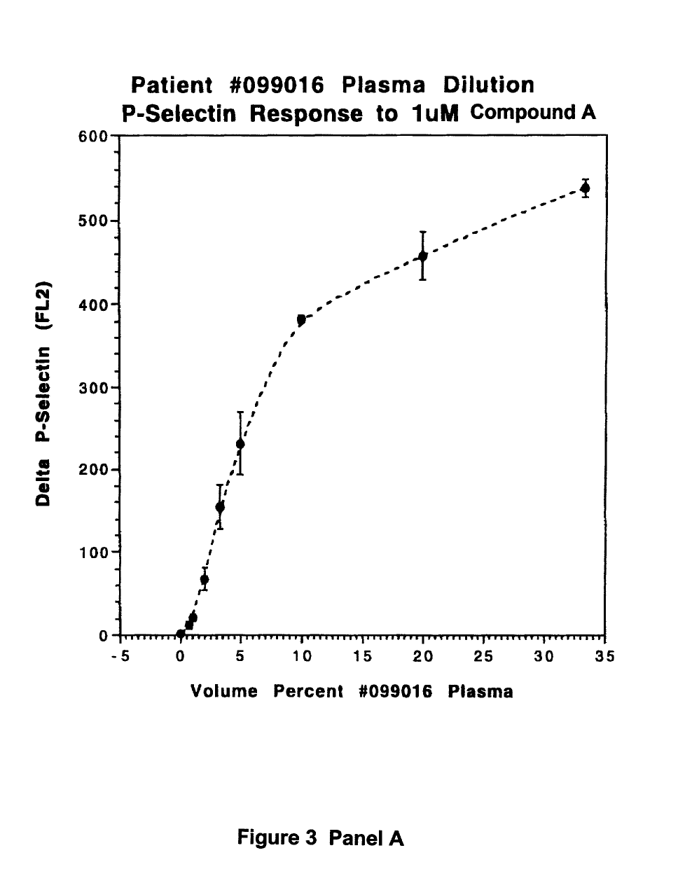

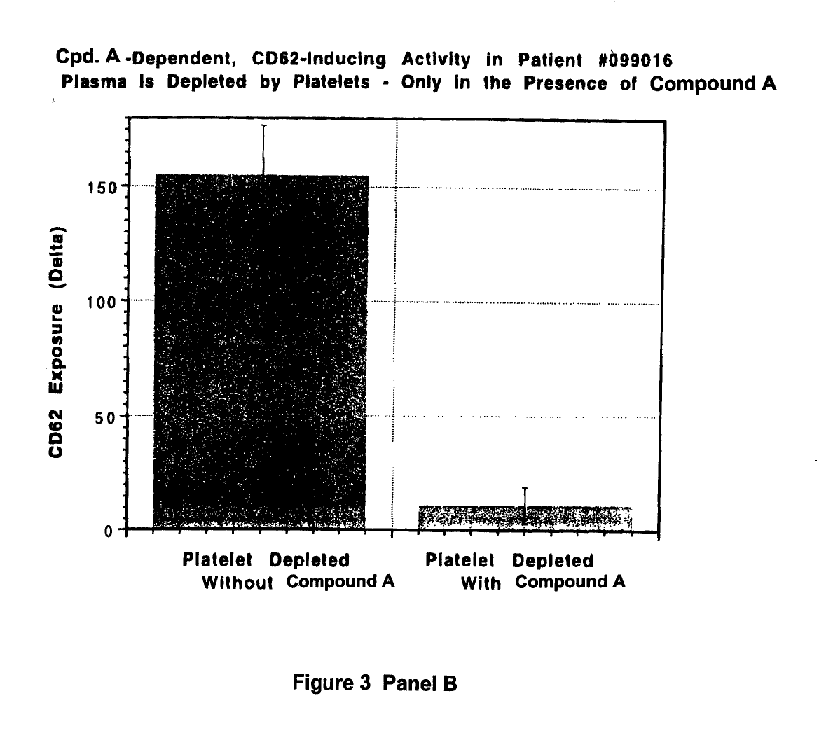

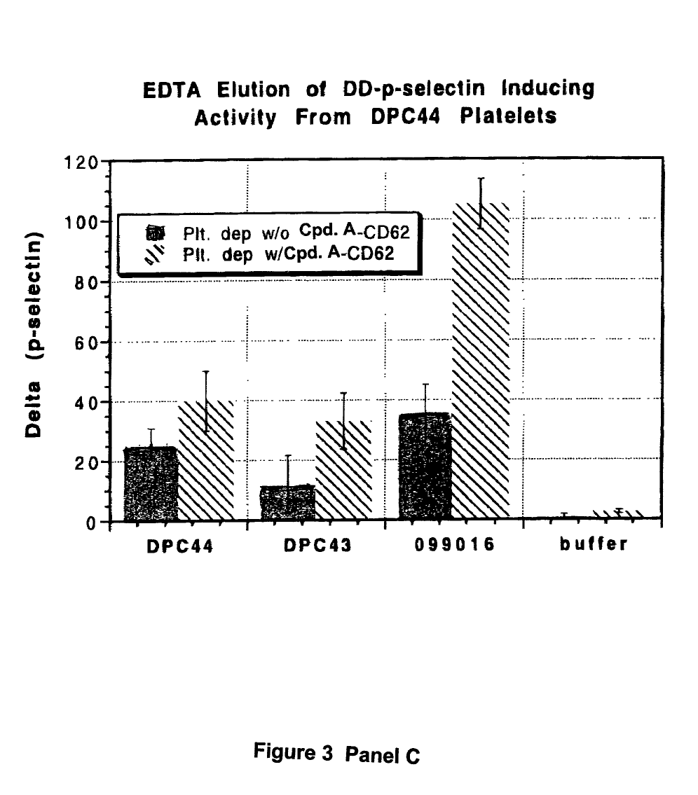

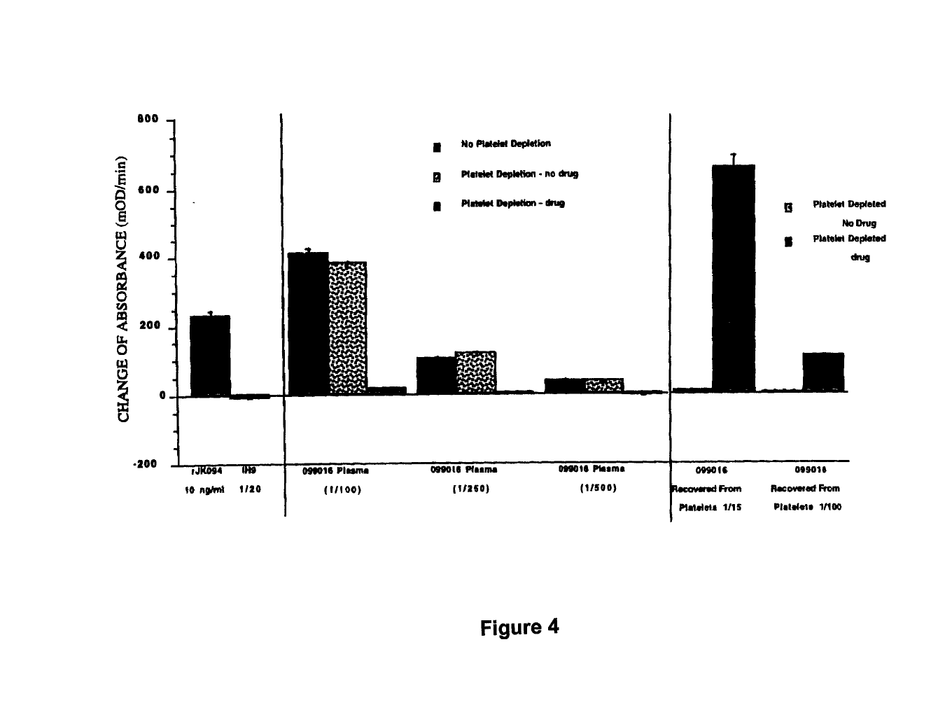

Thrombocytopenic patient plasma was processed as described in Example 1 and Delta fluorescence is shown (FIG. 3, panel A) as a function of volume percent patient plasma. After treatment of 099016 plasma with platelets in the presence and in the absence of compound A, samples were evaluated in the DDPASFCA at a 1/3 dilution using fresh donor PRP. As shown in panel B. prior treatment of 099016 with donor platelets resulted in no loss of detectable DDPAS, whereas prior treatment with donor platelets in the presence of Compound A specifically depleted the DDPAS. This shows the GPIIb/IIIa-antagonist dependent nature of this DDPAS. Analysis of the EDTA elutant from platelets (DDPASFCA-negative donor plasmas DPC43 and DPC44 were similarly processed) treated with 099016 plasma without Compound A showed elevated DDPAS only from the recovered platelet eluant from platelets treated with 099016 plasma with Compound A (panel C), (see commonly-owned pending U.S. patent application Ser. No. 09/237061, filed Jan. 26, 1999, the contents of which are herein incorporated by reference).

FIG. 4: Specific distribution and recovery of thrombocytopenic patient 099016 DDABs onto platelets by Compound A (Example 10)

Thrombocytopenic patient plasma was processed as described in Example 9. After treatment of 099016 plasma with platelets in the presence and in the absence of Compound A, samples were evaluated in the DDAB ELISA (see commonly-owned pending U.S. patent application Ser. No. 09/237061, filed Jan. 26, 1999, the contents of which are herein incorporated by reference) at 3 dilutions (1/100; 1/250 and 1/500) for residual DDAB. Murine JK094 was used as a positive control for the ELISA. Treatment of 099016 plasma with donor platelets resulted in no loss of detectable DDAB, whereas treatment with donor platelets in the presence of Compound A specifically depleted the DDAB. This shows the drug-dependent nature of this anti-platelet antibody. ELISA analysis of the EDTA elutants from platelets treated with 099016 plasma without Compound A were devoid of DDAB, while EDTA eluants from platelets treated with 099016 plasma in the presence of Compound A contain a detectable DDAB, thus illustrating the drug-dependent nature of this anti-platelet antibody.

### DETAILED DESCRIPTION OF THE INVENTION

The present invention provides procedures to identify patients at risk for disease states mediated by treatment with integrin antagonists/agonists. This invention provides procedures to identify patients at risk for integrin antagonist/agonist mediated disease states prior to treatment and during treatment. The present invention provides assays and methods useful for the detection in a patient bodily fluid sample of drug-dependent platelet activating substances (DDPASs) and drug-dependent antibodies (DDAD) that recognize an integrin in the presence of an integrin antagonist/agonist. The present invention cannot differentiate between DDPASs that are DDABs and DDPASs that are not DDABs. The present invention provides sensitive, specific and easy-to-use assays which may be used in conjunction with integrin antagonist/agonist treatment, such assays being capable of detection of low levels of integrin antagonist/antagonist DDABs and DDPASs which may be present in an individual prior to the administration of an integrin antagonist/antagonist and/or for the detection of developing DDPASs and DDABs following treatment with the integrin antagonist/agonist.

An embodiment of the invention provides assays and methods for the detection in a patient bodily fluid sample of activating DDABs that recognize the platelet integrin GPIIb/IIIa in the presence of a GPIIb/IIIa antagonist. The present assays may be used to identify patients at risk of developing GPIIb/IIIa antagonist-induced thrombocytopenia/thromboembolic disease and/or to identify patients who are not at risk of developing GPIIb/IIIa antagonist-induced thrombocytopenia/thromboembolic disease.

The present invention provides methods and assays useful for the detection, in patient body fluid samples, of DDPASs that recognize an integrin. The present invention provides sensitive, specific and easy-to-use assays which may be used in patients to elucidate the involvement of DDPASs to integrins in the disease state, such assays being capable of detecting low levels of integrin directed DDPASs. These DDPASs may be present in patients, blood, body fluids, and tissues without drug therapy. Typical examples include activating auto-antibodies directed to platelet surface antigens, specifically GPIIb/IIIa, which can be encountered in patients with idiopathic thrombocytopenic purpura. In addition, such assays are capable of detecting low levels of activating DDABs directed to integrins and may include antibodies directed to GPIIb/IIIa on the platelet surface, on megakaryocytes or their progenitor cells. These DDPASs may be present in an individual prior to administration of drug therapy, including treatment with integrin antagonists/agonists, and may increase or develop following treatment with drugs.

An embodiment of the invention provides assays and methods for the detection, in patient body fluids, of DDPASs that recognize the platelet integrin GPIIb/IIIa. These DDPASs may arise spontaneously, upon treatment with GPIIb/IIIa antagonists, or other drugs. The present assays and procedures may be used to identify patients at risk of developing thrombocytopenia/thromboembolic complications due to antibodies to GPIIb/IIIa and to identify those who are not at risk to develop these antibodies. The procedures may be used to identify patients at risk of developing GPIIb/IIIa antagonist-dependent DDPASs.

Integrin directed DDPASs may be obtained from, for example, whole blood from individuals that exhibit thrombocytopenia/thromboembolic complications, from untreated individuals having pre-existing antibodies or from treated individuals that develop DDPASs after administration of integrin antagonists/agonist or other medications.

The present invention provides methods for the identification of patients with pre-existing or developing antibody titers to DDPASs directed to GPIIb/IIIa that are at increased risk of developing thrombocytopenia/thromboembolic complications within the initial phase of treatment.

The present invention also provides a method of using a chimeric antibody composition, which recognizes an integrin bound with an integrin agonist/antagonist, as a positive control in DDAB and/or DDPAS assays, (see commonly-owned pending U.S. patent application Ser. No. 09/237061, filed Jan. 26, 1999, the contents of which are herein incorporated by reference).

An embodiment of the invention provides a flow cytometry assay using human platelets and certain GPIIb/IIIa antagonists. The GPIIb/IIIa drug-dependent platelet activating substance flow cytometry assay (herein referred to as DDPASFCA) of the present invention detects pre-existing GPIIb/IIIa Drug-Dependent Platelet Activating Substances (DDPASs) (i.e., DDPASs which are pre-existing in the patient prior to the patient being administered the GPIIb/IIIa antagonist).

The GPIIb/IIIa DDPASFCA of the present invention also detects GPIIb/IIIa DDPASs for which a titer develops following the GPIIb/IIIa antagonist being administered to the patient, such GPIIb/IIIa DDPASs being potentiated by the presence of GPIIb/IIIa antagonists. The present assays and methods may be used to identify individuals having GPIIb/IIIa antagonist-induced DDPASs and may be used to exclude, terminate, and/or change therapeutic modalities with GPIIb/IIIa antagonists prior to the onset of thrombocytopenia/thromboembolic complications.

It has been found in the present invention that use of different GPIIb/IIIa antagonists in the GPIIb/IIIa DDPASFCA detects different DDPASs. Different GPIIb/IIIa antagonists in the GPIIb/IIIa DDPASFCA differ in their ability to induce the exposure of CD62 in a patient. Thus the present assays may be employed to identify integrin antagonists/agonists which may be less likely to induce platelet activation.

GPIIb/IIIa DDPASs may be obtained from, for example, plasma samples from individuals that exhibit thrombocytopenia/thromboembolic complications, from untreated individuals having preexisting DDPASs or from treated individuals that develop DDPASs after administration of a GPIIb/IIIa antagonist. In addition, GPIIb/IIIa DDPASs may be obtained from an individual or organism immunized with GPIIb/IIIa in the presence or absence of a GPIIb/IIIa antagonists. The assays of the present invention can be used to rapidly identify such DDPASs.

The assays of the present invention are also useful for identifying integrin antagonists/agonists that inhibit the integrin receptor but do not potentiate the platelet activity of platelet activating substances and are therefore less likely to potentiate a DDPAS response.

An embodiment of the present invention provides a method for detecting drug-dependent platelet activating substances in a subject which recognize an integrin bound with an integrin antagonist/agonist comprising:

(a) incubating platelets with one or more selected integrin antagonists/agonists, to form a complex between integrin and the selected integrin antagonist/agonist;

(b) incubating the platelet:integrin antagonist/agonist mixture of step (a) with a sample containing a DDPAS from the subject;

(c) incubating the platelet:integrin antagonist/agonist mixture of step (b) with a labeled secondary anti-human CD62 antibody, to form a complex between the labeled secondary anti-human CD62 and CD62 on the platelet surface; and

(d) detecting the labeled secondary antibody.

A preferred embodiment provides the integrin is GPIIb/IIIa.

A preferred embodiment provides the selected integrin antagonist of step (a) is selected from one or more of the following compounds or an active metabolite form thereof:

2(S)-[(n-butoxycarbonyl)amino]-3-[[[3-[4-(aminoiminomethyl)phenyl]isoxazolin-5(R)-yl]methylcarbonyl]amino]propionic acid;

2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;

2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid; and

5-[2-(piperdin-4-yl)ethyl]thieno[2,3-b]thiophene-2-N-(3-2(S)-(3-pyridinylsulfonylamino)propionic acid]carboxamide.

A preferred embodiment provides the labeled secondary anti-human antibody is an anti-human CD62 antibody conjugated with an enzyme or an anti-human CD62 antibody conjugated with a fluorescent label.

A preferred embodiment provides the enzyme is horseradish peroxidase.

A preferred embodiment provides the fluorescent label is phycoerythrin or fluorescein or a derivative thereof.

A preferred embodiment provides the sample containing a DDPAS is plasma obtained from the subject.

Another embodiment of the present invention provides a method for identifying a subject having risk of developing thrombocytopenia/thromboembolic complications during treatment with an integrin antagonist/agonist, wherein platelets are selected from a platelet rich plasma (PRP) from the subject, PRP from the subject diluted with plasma from the subject, or PRP from a healthy human donor diluted with plasma from the subject, comprising:

(a) incubating platelets with one or more selected integrin antagonists/agonists to form a complex between integrin and the selected integrin antagonist/agonist;

(b) incubating the platelet:integrin antagonist/agonist mixture of step (a) with a labeled secondary anti-human CD62 antibody, to form a complex between the labeled secondary anti-human CD62 antibody and CD62 on the platelet surface;

(c) measuring the amount of formation of the complex between the labeled secondary anti-human CD62 antibody and CD62 on the platelet surface of step (b), by detection of the labeled secondary anti-human CD62 antibody label; and

(d) comparing the amount of formation of the complex between the labeled secondary anti-human CD62 antibody and CD62 on the platelet surface of step (c) with the amount of such complex formed when steps (b), (c), and (d) are carried out and step (a) is omitted.

A preferred embodiment provides the sample containing DDPAS is obtained from the subject and the method is performed prior to treatment of the subject with an integrin antagonist/agonist.

A preferred embodiment provides the sample containing DDPAS is obtained from the subject and the method is performed concurrently with treatment of the subject with an integrin antagonist/agonist.

A preferred embodiment provides the selected integrin antagonists/agonists of step (a) comprise the active form or active metabolite of the integrin antagonist/agonist which is used to treat the subject.

A preferred embodiment provides the selected integrin antagonist of step (a) is selected from one or more of the following compounds or an active metabolite form thereof:

2(S)-[(n-butoxycarbonyl)amino]-3-[[[3-[4-(aminoiminomethyl)phenyl]isoxazolin-5(R)-yl]methylcarbonyl]amino]propionic acid;

2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;

2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid; and

5-[2-(piperdin-4-yl)ethyl]thieno[2,3-b]thiophene-2-N-(3-2(S)-(3-pyridinylsulfonylamino)propionic acid]carboxamide.

Another embodiment of the present invention provides a method of treating a subject with an integrin antagonist/agonist, comprising:

(a) performing the above method wherein the sample containing DDPAS is obtained from the subject and the method is performed prior to treating the subject with the integrin antagonist/agonist;

(b) administering to the subject an effective amount of a pharmaceutical composition comprising the integrin antagonist/agonist; and

(c) performing the above method wherein the sample containing DDPAS is obtained from the subject and the method is performed concurrently with treatment of the subject with the integrin antagonist/agonist.

A preferred embodiment provides the subject is treated with an integrin antagonist selected from one or more of the following compounds:

2(S)-[(n-butoxycarbonyl)amino]-3-[[[3-[4-(aminoiminomethyl)phenyl]isoxazolin-5(R)-yl]methylcarbonyl]amino]propionic acid or the methyl ester thereof;

2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;

2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid;

5-[2-(piperdin-4-yl)ethyl]thieno[2,3-b]thiophene-2-N-(3-2(S)-(3-pyridinylsulfonylamino)propionic acid]carboxamide.

Another embodiment of the present invention provides a diagnostic flow cytometry kit, comprising: at least one selected integrin antagonist/agonist and a secondary labeled anti-human CD62 antibody to be used in conjunction with a source of platelets.

Another method of the present invention provides a method of determining whether a selected integrin antagonist/agonist potentiates the exposure of CD62 in a subject who's blood recognizes an integrin bound with an integrin antagonist/agonist, comprising:

(a) incubating platelets with one or more selected integrin antagonists/agonists to form a complex between integrin and the selected integrin antagonist/agonist;

(b) incubating the platelet:integrin antagonist/agonist mixture of step (a) with a sample containing a DDPAS from the subject; and

(c) incubating the platelet:integrin antagonist/agonist mixture of step (b) with a labeled secondary anti-human CD62 antibody, to form a complex between the labeled secondary anti-human CD62 and CD62 on the platelet surface; and

(d) detecting the labeled secondary antibody.

It is preferred in the above methods that the sample containing the DDPAS is citrated plasma obtained from the subject.

The term “integrin” as used herein refers to any of the many cell surface receptor proteins, also referred to as adhesion protein receptors, which have been identified which bind to extracellular matrix ligands or other cell adhesion protein ligands thereby mediating cell-cell and cell-matrix adhesion processes. The integrins are encoded by genes belonging to a gene superfamily and are typically composed of heterodimeric transmembrane glycoproteins containing α and β-subunits. Integrin subfamilies contain a common β-subunit combined with different α-subunits to form adhesion protein receptors with different specificities.

The integrin glycoprotein IIb/IIIa (referred to herein as GPIIb/IIIa or IIb/IIIa or the fibrinogen receptor) is the membrane protein mediating platelet aggregation. GPIIb/IIIa in activated platelets is known to bind four soluble RGD-containing adhesive proteins, namely fibrinogen, von Willebrand factor, fibronectin, and vitronectin. In addition to GPIIb/IIIa, a number of other integrin cell surface receptors have been identified, for example, αvβ3, α4β1 and α5β1.

The term “antibody” as used herein includes antibody from a monoclonal or polyclonal source which is produced in response to an antigen, as well as fragments, chimeric forms, altered forms and derivatives of such antibody, as well as chemically and recombinantly produced forms thereof. The term “anti-human antibody” as used herein refers to an antibody which recognizes and binds to human immunoglobulin. The term “platelet activating substances” as used herein includes, but is not limited to, ADP, platelet activating antibodies, drug-dependent platelet activating antibodies, and other activators of the basic platelet reaction including thrombin, epinephrine, collagen, arachidonate and the thrombin receptor activating peptide, TRAP.

The term “JK094” as used herein refers to a chimeric monoclonal antibody specific for the gel-shifted form of GPIIb/IIIa, whose cloning, PCR recombination, production, purification and characterization are disclosed in pending, commonly owned U.S. patent application Ser. No. 09/237061, the contents of which are incorporated herein by reference.

As used herein, the term “anti-human detectable antibody” refers to an anti-human antibody that can be detected directly or indirectly by a variety of means known in the art. The anti-human detectable antibody is preferably a labeled secondary anti-human antibody. As used herein, the term “labeled secondary anti-human antibody” refers to an anti-human antibody which is labeled or conjugated or otherwise associated with a label or detectable marker which can be detected directly or indirectly by a variety of means known in the art. The labeled secondary anti-human antibody preferably contains a fluorescent label or an enzyme label, such as horseradish peroxidase, which induces a detectable reaction when exposed to a substrate that is acted upon by the enzyme.

The source of the DDPASs sample to be tested in the assays of the present invention may be any bodily fluid or tissue or cells containing such DDPASs, with the preferred source of such DDPASs sample being blood or plasma.

The term “integrin antagonists” as referred to herein (also referred to herein as integrin inhibitors) includes compounds (including proteins, peptides, peptideomimetic compounds and other small molecule compounds) which act as inhibitors of the binding of the integrin protein to endogenous protein ligands of such integrin. The term “integrin agonists”, as referred to herein, includes compounds which act as stimulators of the binding of the integrin protein to endogenous proteins ligands of such integrin. Preferred integrin inhibitors used in the present invention are RGD-peptidomimetic compounds. As used herein, the term “RGD-peptidomimetic compounds” refers to chemical compounds which bind to the RGD-binding region of the integrin and which block RGD-mediated binding of one or more adhesive proteins to such integrin. Preferred in the present invention are antagonists of the GPIIb/IIIa integrin.

Representative integrin antagonist compounds, including GPIIb/IIIa antagonists are disclosed in the following patents and patent applications, which are incorporated herein by reference: PCT Patent Application 95/14683; PCT Patent Application 95/32710; U.S. Pat. Nos. 5,334,596; 5,276,049; 5,281,585; European Patent Application 478,328; European Patent Application 478,363; European Patent Application 512,831; PCT Patent Application 94/08577; PCT Patent Application 94/08962; PCT Patent Application 94/18981; PCT Patent Application 93/16697; Canada Patent Application 2,075,590; PCT Patent Application 93/18057; European Patent Application 445,796; Canada Patent Application 2,093,770; Canada Patent Application 2,094,773; Canada Patent Application 2,101,179; Canada Patent Application 2,074,685; Canada Patent Application 2,094,964; Canada Patent Application 2,105,934; Canada Patent Application 2,114,178; Canada Patent Application 2,116,068; European Patent Application 513,810; PCT Patent Application 95/06038; European Patent Application 381,033; PCT Patent Application 93/07867; and PCT Patent Application 94/02472.

Integrin antagonists useful in the present invention are compounds, or active metabolites thereof, selected from:

N³-[2-{3-(4-formamidinophenyl)-isoxazolin-5(R,S)-yl}-acetyl]-N2-(phenylsulfonyl)-2,3-(S)-diaminopropanoic acid;

N³-[2-{3-(4-formamidinophenyl)-isoxazolin-5(R,S)-yl}-acetyl]-N2-(4-methyl-phenyl-sulfonyl)-2,3-(S)-diaminopropanoic acid;

N³-[2-{3-(4-formamidinophenyl)-isoxazolin-5(R,S)-yl}-acetyl]-N2-(butanesulfonyl)-2,3-(S)-diaminopropanoic acid;

N³-[2-{3-(4-formamidinophenyl)-isoxazolin-5(R,S)-yl}-acetyl]-N2-(propanesulfonyl)-2,3-(S)-diaminopropanoic acid;

N³-[2-{3-(4-formamidinophenyl)-isoxazolin-5(R,S)-yl}-acetyl]-N2-(ethanesulfonyl)-2,3-(S)-diaminopropanoic acid;

N³-[2-{3-(4-formamidinophenyl)-isoxazolin-5(R,S)-yl}-acetyl]-N2-(methyloxycarbonyl)-2,3-(S)-diaminopropanoic acid;

N³-[2-{3-(4-formamidinophenyl)-isoxazolin-5(R,S)-yl}-acetyl]-N2-(ethyloxycarbonyl)-2,3-(S)-diaminopropanoic acid;

N³-[2-{3-(4-formamidinophenyl)-isoxazolin-5(R,S)-yl}-acetyl]-N2-(1-propyloxycarbonyl)-2,3-(S)-diaminopropanoic acid;

N³-[2-{3-(4-formamidinophenyl)-isoxazolin-5(R,S)-yl}-acetyl]-N2-(2-propyloxycarbonyl)-2,3-(S)-diaminopropanoic acid;

N³-[2-{3-(4-formamidinophenyl)-isoxazolin-5(R,S)-yl}-acetyl]-N2-(n-butyloxycarbonyl)-2,3-(S)-diaminopropanoic acid;

N³-[2-{3-(4-formamidinophenyl)-isoxazolin-5(R)-yl}-acetyl]-N2-(n-butyloxycarbonyl)-2,3-(S)-diaminopropanoic acid;

N³-[2-{3-(4-formamidinophenyl)-isoxazolin-5(S)-yl}-acetyl]-N2-(n-butyloxycarbonyl)-2,3-(S)-diaminopropanoic acid;

N³-[2-{3-(4-formamidinophenyl)-isoxazolin-5(R)-yl}-acetyl]-N2-(n-butyloxycarbonyl)-2,3-(R)-diaminopropanoic acid;

N³-[2-{3-(4-formamidinophenyl)-isoxazolin-5(S)-yl}-acetyl]-N2-(n-butyloxycarbonyl)-2,3-(R)-diaminopropanoic acid;

N³-[2-{3-(4-formamidinophenyl)-isoxazolin-5(R,S)-yl}-acetyl]-N2-(2-butyloxycarbonyl)-2,3-(S)-diaminopropanoic acid;

N³-[2-{3-(4-formamidinophenyl)-isoxazolin-5(R,S)-yl}-acetyl]-N2- (1-(2-methyl) -propyloxycarbonyl) -2,3-(S)-diaminopropanoic acid;

N³-[2-{3-(4-formamidinophenyl)-isoxazolin-5(R,S)-yl}-acetyl]-N2-(2-(2-methyl)-propyloxycarbonyl)-2,3-(S)-diaminopropanoic acid;

N³-[2-{3-(4-formamidinophenyl)-isoxazolin-5(R,S)-yl}-acetyl]-N2-(benzyloxycarbonyl)-2,3-(S)-diaminopropanoic acid;

N³-[2-{3-(4-formamidinophenyl)-isoxazolin-5(R)-yl}-acetyl]-N2-(benzyloxycarbonyl)-2,3-(S)-diaminopropanoic acid;

N³-[2-{3-(4-formamidinophenyl)-isoxazolin-5(S)-yl}-acetyl]-N2-(benzyloxycarbonyl)-2,3-(S)-diaminopropanoic acid;

N³-[2-{3-(4-formamidinophenyl)-isoxazolin-5(R,S)-yl}-acetyl]-N2-(4-methylbenzyloxycarbonyl)-2,3-(S)-diaminopropanoic acid;

N³-[2-{3-(4-formamidinophenyl)-isoxazolin-5(R,S)-yl}-acetyl]-N2-(4-methoxybenzyloxycarbonyl)-2,3-(S)-diaminopropanoic acid;

N³-[2-{3-(4-formamidinophenyl)-isoxazolin-5(R,S)-yl}-acetyl]-N2-(4-chlorobenzyloxycarbonyl)-2,3-(S)-diaminopropanoic acid;

N³-[2-{3-(4-formamidinophenyl)-isoxazolin-5(R,S)-yl}-acetyl]-N2-(4-bromobenzyloxycarbonyl)-2,3-(S)-diaminopropanoic acid;

N³-[2-{3-(4-formamidinophenyl)-isoxazolin-5(R,S)-yl}-acetyl]-N2-(4-fluorobenzyloxycarbonyl)-2,3-(S)-diaminopropanoic acid;

N³-[2-{3-(4-formamidinophenyl)-isoxazolin-5(R,S)-yl}-acetyl]-N2-(4-phenoxybenzyloxycarbonyl)-2,3-(S)-diaminopropanoic acid;

N³-[2-{3-(4-formamidinophenyl)-isoxazolin-5(R,S)-yl}-acetyl]-N2-(2-(methyloxyethyl)-oxycarbonyl)-2,3-(S)-diaminopropanoic acid;

N³-[2-{3-(4-formamidinophenyl)-isoxazolin-5(R,S)-yl}-acetyl]-N2-(2-pyridinylcarbonyl)-2,3-(S)-diaminopropanoic acid;

N³-[2-{3-(4-formamidinophenyl)-isoxazolin-5(R,S)-yl}-acetyl]-N2-(3-pyridinylcarbonyl)-2,3-(S)-diaminopropanoic acid;

N³-[2-{3-(4-formamidinophenyl)-isoxazolin-5(R,S)-yl}-acetyl]-N2-(4-pyridinyl-carbonyl)-2,3-(S)-diaminopropanoic acid;

N³-[2-{3-(4-formamidinophenyl)-isoxazolin-5(R,S)-yl}-acetyl]-N2-(2-(2-pyridinyl)-acetyl)-2,3-(S)-diaminopropanoic acid;

N³-[2-{3-(4-formamidinophenyl)-isoxazolin-5(R,S)-yl}-acetyl]-N2-(2-(3-pyridinyl)-acetyl)-2,3-(S)-diaminopropanoic acid;

N³-[2-{3-(4-formamidinophenyl)-isoxazolin-5(R,S)-yl}-acetyl]-N2-(2-(4-pyridinyl)-acetyl)-2,3-(S)-diaminopropanoic acid;

N³-[2-{3-(4-formamidinophenyl)-isoxazolin-5(R,S)-yl}-acetyl]-N2-(2-pyridyl-methyloxycarbonyl)-2,3-(S)-diaminopropanoic acid;

N³-[2-{3-(4-formamidinophenyl)-isoxazolin-5(R,S)-yl}-acetyl]-N2-(3-pyridyl-methyloxycarbonyl)-2,3-(S)-diaminopropanoic acid;

N³-[2-{3-(4-formamidinophenyl)-isoxazolin-5(R,S)-yl}-acetyl]-N2-(4-pyridyl-methyloxycarbonyl)-2,3-(S)-diaminopropanoic acid;

N³-[2-{3-(4-formamidinophenyl)-isoxazolin-5(R,S)-yl}-acetyl]-N2-(4-butyloxyphenylsulfonyl)-2,3-(S)-diaminopropanoic acid;

N³-[2-{3-(4-formamidinophenyl)-isoxazolin-5(R,S)-yl}-acetyl]-N2-(2-thienylsulfonyl)-2,3-(S)-diaminopropanoic acid;

N³-[2-{3-(4-formamidinophenyl)-isoxazolin-5(R,S)-yl}-acetyl]-N2-(3-methylphenylsulfonyl)-2,3-(R,S)-diaminopropanoic acid;

N³-[2-{3-(4-formamidinophenyl)-isoxazolin-5(R,S)-yl}-acetyl]-N2-(3-methylphenylsulfonyl)-2,3-(S)-diaminopropanoic acid;

N³-[2-{3-(4-formamidinophenyl)-isoxazolin-5(R,S)-yl}-acetyl]-N2-(3-methylphenylsulfonyl)-2,3-(R)-diaminopropanoic acid;

N³-[2-{3-(4-formamidinophenyl)-isoxazolin-5(R)-yl}-acetyl]-N2-(3-methylphenylsulfonyl)-2,3-(S)-diaminopropanoic acid;

N³-[2-{3-(4-formamidinophenyl)-isoxazolin-5(S)-yl}-acetyl]-N2-(3-methylphenylsulfonyl)-2,3-(S)-diaminopropanoic acid;

N³-[2-{3-(4-formamidinophenyl)-isoxazolin-5(S)-yl}-acetyl]-N2-(3-methylphenylsulfonyl)-2,3-(R)-diaminopropanoic acid;

N³-[2-{3-(4-formamidinophenyl)-isoxazolin-5(R)-yl}-acetyl]-N2-(3-methylphenylsulfonyl)-2,3-(R)-diaminopropanoic acid;

N³-[2-{3-(4-formamidinophenyl)-isoxazolin-5(R,S)-yl}-acetyl]-N2-(4-iodophenylsulfonyl)-2,3-(S)-diaminopropanoic acid;

N³-[2-{3-(4-formamidinophenyl)-isoxazolin-5(R,S)-yl}-acetyl]-N2-(3-trifluoromethylphenylsulfonyl)-2,3-(S)-diaminopropanoic acid;

N³-[2-{3-(4-formamidinophenyl)-isoxazolin-5(R,S)-yl}-acetyl]-N2- (3-chlorophenylsulfonyl)-2,3-(S) -diaminopropanoic acid;

N³-[2-{3-(4-formamidinophenyl)-isoxazolin-5(R,S)-yl}-acetyl]-N2- (3-2-methoxycarbonylphenylsulfonyl) -2,3-(S)-diaminopropanoic acid;

N³-[2-{3-(4-formamidinophenyl)-isoxazolin-5(R,S)-yl}-acetyl]-N2-(2,4,6-trimethylphenylsulfonyl) -2,3-(S)-diaminopropanoic acid;

N³-[2-{3-(4-formamidinophenyl)-isoxazolin-5(R,S)-yl}-acetyl]-N2-(2-chlorophenylsulfonyl)-2,3-(S)-diaminopropanoic acid;

N³-[2-{3-(4-formamidinophenyl)-isoxazolin-5(R,S)-yl}-acetyl]-N2-(4-trifluoromethylphenylsulfonyl)-2,3-(S)-diaminopropanoic acid;

N³-[2-{3-(4-formamidinophenyl)-isoxazolin-5(R,S)-yl}-acetyl]-N2-(2-trifluoromethylphenylsulfonyl)-2,3-(S)-diaminopropanoic acid;

N³-[2-{3-(4-formamidinophenyl)-isoxazolin-5(R,S)-yl}-acetyl]-N2-(2-fluorophenylsulfonyl)-2,3-(S)-diamninopropanoic acid;

N³-[2-{3-(4-formamidinophenyl)-isoxazolin-5(R,S)-yl}-acetyl]-N2-(4-fluorophenylsulfonyl)-2,3-(S)-diamninopropanoic acid;

N³-[2-{3-(4-formamidinophenyl)-isoxazolin-5(R,S)-yl}-acetyl]-N2-(4-methoxyphenylsulfonyl)-2,3-(S)-diaminopropanoic acid;

N³-[2-{3-(4-formamidinophenyl)-isoxazolin-5(R,S)-yl}-acetyl]-N2-(2,3,5,6-tetramethylphenylsulfonyl)-2,3-(S)-diamninopropanoic acid;

N³-[2-{3-(4-formamidinophenyl)-isoxazolin-5(R,S)-yl}-acetyl]-N2-(4-cyanophenylsulfonyl)-2,3-(S)-diaminopropanoic acid;

N³-[2-{3-(4-formamidinophenyl)-isoxazolin-5(R,S)-yl}-acetyl]-N2-(4-chlorophenylsulfonyl)-2,3-(S)-diaminopropanoic acid;

N³-[2-{3-(4-formamidinophenyl)-isoxazolin-5(R,S)-yl}-acetyl]-N2-(4-propylphenylsulfonyl)-2,3-(S)-diaminopropanoic acid;

N³-[2-{3-(4-formamidinophenyl)-isoxazolin-5(R,S)-yl}-acetyl]-N2-(2-phenylethylsulfonyl)-2,3-(S)-diaminopropanoic acid;

N³-[2-{3-(4-formamidinophenyl)-isoxazolin-5(R,S)-yl}-acetyl]-N2-(4-isopropylphenylsulfonyl)-2,3-(S)-diaminopropanoic acid;

N³-[2-{3-(4-formamidinophenyl)-isoxazolin-5(R,S)-yl}-acetyl]-N2-(3-phenylpropylsulfonyl)-2,3-(S)-diaminopropanoic acid;

N³-[2-{3-(4-formamidinophenyl)-isoxazolin-5(R,S)-yl}-acetyl]-N2-(3-pyridylsulfonyl)-2,3-(S)-diaminopropanoic acid;

N³-[2-{3-(4-formamidinophenyl)-isoxazolin-5(R,S)-yl}-acetyl]-N2-(phenylaminosulfonyl)-2,3-(S)-diaminopropanoic acid;

N³-[2-{3-(4-formamidinophenyl)-isoxazolin-5(R,S)-yl}-acetyl]-N2-(benzylaminosulfonyl)-2,3-(S)-diaminopropanoic acid;

N³-[2-{3-(4-formamidinophenyl)-isoxazolin-5(R,S)-yl}-acetyl]-N2-(dimethylaminosulfonyl)-2,3-(S)-diaminopropanoic acid;

N³-[2-{3-(2-fluoro-4-formamidinophenyl)-isoxazolin-5(R,S)-yl}-acetyl]-N2-(3-methylphenylsulfonyl)-2,3-(S)-diaminopropanoic acid;

N³-[2-{3-(2-formamidino-5-pyridinyl)-isoxazolin-5(R,S)-yl}-acetyl]-N2-(n-butyloxycarbonyl)-2,3-(S)-diaminopropanoic acid;

N³-[2-{3-(2-formamidino-5-pyridinyl)-isoxazolin-5(R,S)-yl}-acetyl]-N2-(3-methylphenylsulfonyl)-2,3-(S)-diaminopropanoic acid;

N³-[2-{3-(3-formamidino-6-pyridinyl)-isoxazolin-5(R,S)-yl}-acetyl]-N2-(n-butyloxycarbonyl)-2,3-(S)-diaminopropanoic acid;

N³-[2-{3-(3-formamidino-6-pyridinyl)-isoxazolin-5(R,S)-yl}-acetyl]-N2-(3-methylphenylsulfonyl)-2,3-(S)-diaminopropanoic acid;

N³-[2-{3-(4-formamidinophenyl)-isoxazolin-5(R,S)-yl}-acetyl]-N2-(phenylaminocarbonyl)-2,3-(S)-diaminopropanoic acid;

N³-[2-{3-(4-formamidinophenyl)-isoxazolin-5(R,S)-yl}-acetyl]-N2-(4-fluorophenylaminocarbonyl)-2,3-(S)-diaminopropanoic acid;

N³-[2-{3-(4-formamidinophenyl)-isoxazolin-5(R,S)-yl}-acetyl]-N2-(1-naphthylaminocarbonyl)-2,3-(S)-diaminopropanoic acid;

N³-[2-{3-(4-formamidinophenyl)-isoxazolin-5(R,S)-yl}-acetyl]-N2-(benzylaminocarbonyl)-2,3-(S)-diaminopropanoic acid;

N³-[2-{3-(4-formamidinophenyl)-isoxazolin-5(R,S)-yl}-acetyl]-N2-(3-bromo-2-thienylsulfonyl)-2,3-(S)-diaminopropanoic acid;

N³-[2-{3-(4-formamidinophenyl)-isoxazolin-5(R,S)-yl}-acetyl]-N2-(3-methyl-2-benzothienylsulfonyl)-2,3-(S)-diaminopropanoic acid;

N³-[2-{3-(4-formamidinophenyl)-isoxazolin-5(R,S)-yl}-acetyl]-N2-(isobutyloxycarbonyl)-2,3-(S)-diaminopropanoic acid;

N³-[2-{3-(4-formamidinophenyl)-isoxazolin-5(R)-yl}-acetyl]-N2-(isobutyloxycarbonyl)-2,3-(S)-diaminopropanoic acid;

N³-[2-{3-(4-formamidinophenyl)-isoxazolin-5(S)-yl}-acetyl]-N2-(isobutyloxycarbonyl)-2,3-(S)-diaminopropanoic acid;

N³-[2-{3-(4-formamidinophenyl)-isoxazolin-5(R,S)-yl}-acetyl]-N2-(2-cyclopropylethoxycarbonyl)-2,3-(S)-diaminopropanoic acid;

N³-[2-{3-(4-formamidinophenyl)-isoxazolin-5(R)-yl}-acetyl]-N2-(2-cyclopropylethoxycarbonyl)-2,3-(S)-diaminopropanoic acid;

N³-[2-{3-(4-formamidinophenyl)-isoxazolin-5(S)-yl}-acetyl]-N2-(2-cyclopropylethoxycarbonyl)-2,3-(S)-diaminopropanoic acid;

N³-[2-{3-(4-guanidinophenyl)-isoxazolin-5(R,S)-yl}-acetyl]-N2-(n-butyloxycarbonyl)-2,3-(S)-diaminopropanoic acid;

N³-[2-{3-(4-guanidinophenyl)-isoxazolin-5(R)-yl}-acetyl]-N2-(n-butyloxycarbonyl)-2,3-(S)-diaminopropanoic acid;

N³-[2-{3-(4-guanidinophenyl)-isoxazolin-5(R)-yl}-acetyl]-N2-(3-methylphenylsulfonyl)-2,3-(S)-diaminopropanoic acid;

N³-[2-{5-(4-formamidinophenyl)-isoxazolin-3(R,S)-yl}-acetyl]-N2-(n-butyloxycarbonyl)-2,3-(S)-diaminopropanoic acid ;

or a propionate ester prodrug form of said compound, wherein the hydrogen of the hydroxy group of the diaminopropanoic acid moiety is substituted with a group selected from:

methyl;

ethyl;

isopropyl;

methylcarbonyloxymethyl-;

ethylcarbonyloxymethyl-;

t-butylcarbonyloxymethyl-;

cyclohexylcarbonyloxymethyl-;

1-(methylcarbonyloxy)ethyl-;

1-(ethylcarbonyloxy)ethyl-;

1-(t-butylcarbonyloxy)ethyl-;

1-(cyclohexylcarbonyloxy)ethyl-;

i-propyloxycarbonyloxymethyl-;

cyclohexylcarbonyloxymethyl-;

t-butyloxycarbonyloxymethyl-;

1-(i-propyloxycarbonyloxy)ethyl-;

1-(cyclohexyloxycarbonyloxy)ethyl-;

1-(t-butyloxycarbonyloxy)ethyl-;

dimethylaminoethyl-;

diethylaminoethyl-;

(5-methyl-1,3-dioxacyclopenten-2-on-4-yl)methyl-;

(5-(t-butyl)-1,3-dioxacyclopenten-2-on-4-yl)methyl-;

(1,3-dioxa-5-phenyl-cyclopenten-2-on-4-yl)methyl-;

1-(2-(2-methoxypropyl)carbonyloxy)ethyl-.

Further preferred integrin antagonists useful in the present invention are compounds, or enantiomeric or diasteriomeric forms thereof, or mixtures of enantiomeric or diasteriomeric forms thereof, or active metabolites thereof, and salt forms thereof, selected from:

N³-[2-{3-(4-formamidinophenyl)-isoxazolin-5-yl}-acetyl]-N²-(phenylsulfonyl)-2,3-diaminopropanoic acid;

N³-[2-{3-(4-formamidinophenyl)-isoxazolin-5-yl}-acetyl]-N²-(4-methyl-phenyl-sulfonyl)-2,3-diaminopropanoic acid;

N³-[2-{3-(4-formamidinophenyl)-isoxazolin-5-yl}-acetyl]-N²-(butanesulfonyl)-2,3-diaminopropanoic acid;

N³-[2-{3-(4-formamidinophenyl)-isoxazolin-5-yl}-acetyl]-N²-(propanesulfonyl)-2,3-diaminopropanoic acid;

N³-[2-{3-(4-formamidinophenyl)-isoxazolin-5-yl}-acetyl]-N²-(ethanesulfonyl)-2,3-diaminopropanoic acid;

N³-[2-{3-(4-formamidinophenyl)-isoxazolin-5-yl}-acetyl]-N²-(methyloxycarbonyl)-2,3-diaminopropanoic acid;

N³-[2-{3-(4-formamidinophenyl)-isoxazolin-5-yl}-acetyl]-N²-(ethyloxycarbonyl)-2,3-diaminopropanoic acid;

N³-[2-{3-(4-formamidinophenyl)-isoxazolin-5-yl}-acetyl]-N²-(1-propyloxycarbonyl)-2,3-diaminopropanoic acid;

N³-[2-{3-(4-formamidinophenyl)-isoxazolin-5-yl}-acetyl]-N²-(2-propyloxycarbonyl)-2,3-diaminopropanoic acid;

N³-[2-{3-(4-formamidinophenyl)-isoxazolin-5-yl}-acetyl]-N²-(n-butyloxycarbonyl)-2,3-diaminopropanoic acid;

N³-[2-{3-(4-formamidinophenyl)-isoxazolin-5-yl}-acetyl]-N²-(1-(2-methyl)-propyloxycarbonyl)-2,3-diaminopropanoic acid;

N³-[2-{3-(4-formamidinophenyl)-isoxazolin-5-yl}-acetyl]-N²-(2-(2-methyl)-propyloxycarbonyl)-2,3-diaminopropanoic acid;

N³-[2-{3-(4-formamidinophenyl)-isoxazolin-5-yl}-acetyl]-N²-(benzyloxycarbonyl)-2,3-diaminopropanoic acid;

N³-[2-{3-(4-formamidinophenyl)-isoxazolin-5-yl}-acetyl]-N²-(4-methylbenzyloxycarbonyl)-2,3-diaminopropanoic acid;

N³-[2-{3-(4-formamidinophenyl)-isoxazolin-5-yl}-acetyl]-N²-(4-methoxybenzyloxycarbonyl)-2,3-diaminopropanoic acid;

N³-[2-{3-(4-formamidinophenyl)-isoxazolin-5-yl}-acetyl]-N²-(4-chlorobenzyloxycarbonyl)-2,3-diaminopropanoic acid;

N³-[2-{3-(4-formamidinophenyl)-isoxazolin-5-yl}-acetyl]-N²-(4-bromobenzyloxycarbonyl)-2,3-diaminopropanoic acid;

N³-[2-{3-(4-formamidinophenyl)-isoxazolin-5-yl}-acetyl]-N²-(4-fluorobenzyloxycarbonyl)-2,3-diaminopropanoic acid;

N³-[2-{3-(4-formamidinophenyl)-isoxazolin-5-yl}-acetyl]-N²-(4-phenoxybenzyloxycarbonyl)-2,3-diaminopropanoic acid;

N³-[2-{3-(4-formamidinophenyl)-isoxazolin-5-yl}-acetyl]-N²-(2-(methyloxyethyl)-oxycarbonyl)-2,3-diaminopropanoic acid;

N³-[2-{3-(4-formamidinophenyl)-isoxazolin-5-yl}-acetyl]-N²-(2-pyridinylcarbonyl)-2,3-diaminopropanoic acid;

N³-[2-{3-(4-formamidinophenyl)-isoxazolin-5-yl}-acetyl]-N²-(3-pyridinylcarbonyl)-2,3-diaminopropanoic acid;

N³-[2-{3-(4-formamidinophenyl)-isoxazolin-5-yl}-acetyl]-N²-(4-pyridinyl-carbonyl)-2,3-diaminopropanoic acid;

N³-[2-{3-(4-formamidinophenyl)-isoxazolin-5-yl}-acetyl]-N²-(2-(2-pyridinyl)-acetyl)-2,3-diaminopropanoic acid;

N³-[2-{3-(4-formamidinophenyl)-isoxazolin-5-yl}-acetyl]-N²-(2-(3-pyridinyl)-acetyl)-2,3-diaminopropanoic acid;

N³-[2-{3-(4-formamidinophenyl)-isoxazolin-5-yl}-acetyl]-N²-(2-(4-pyridinyl)-acetyl)-2,3-diaminopropanoic acid;

N³-[2-{3-(4-formamidinophenyl)-isoxazolin-5-yl}-acetyl]-N²-(2-pyridyl-methyloxycarbonyl)-2,3-diaminopropanoic acid;

N³-[2-{3-(4-formamidinophenyl)-isoxazolin-5-yl}-acetyl]-N²-(3-pyridyl-methyloxycarbonyl)-2,3-diaminopropanoic acid;

N³-[2-{3-(4-formamidinophenyl)-isoxazolin-5-yl}-acetyl]-N²-(4-pyridyl-methyloxycarbonyl)-2,3-diaminopropanoic acid;

N³-[2-{3-(4-formamidinophenyl)-isoxazolin-5-yl}-acetyl]-N²-(4-butyloxyphenylsulfonyl)-2,3-diaminopropanoic acid;

N³-[2-{3-(4-formamidinophenyl)-isoxazolin-5-yl}-acetyl]-N²-(2-thienylsulfonyl)-2,3-diaminopropanoic acid;

N³-[2-{3-(4-formamidinophenyl)-isoxazolin-5-yl}-acetyl]-N²-(3-methylphenylsulfonyl)-2,3-diaminopropanoic acid;

N³-[2-{3-(4-formamidinophenyl)-isoxazolin-5-yl}-acetyl]-N²-(4-iodophenylsulfonyl)-2,3-diaminopropanoic acid;

N³-[2-{3-(4-formamidinophenyl)-isoxazolin-5-yl}-acetyl]-N²-(3-trifluoromethylphenylsulfonyl)-2,3-diaminopropanoic acid;

N³-[2-{3-(4-formamidinophenyl)-isoxazolin-5-yl}-acetyl]-N²-(3-chlorophenylsulfonyl)-2,3-diaminopropanoic acid;

N³-[2-{3-(4-formamidinophenyl)-isoxazolin-5-yl}-acetyl]-N²-(2-methoxycarbonylphenylsulfonyl)-2,3-diaminopropanoic acid;

N³-[2-{3-(4-formamidinophenyl)-isoxazolin-5-yl}-acetyl]-N²-(2,4,6-trimethylphenylsulfonyl)-2,3-diaminopropanoic acid;

N³-[2-{3-(4-formamidinophenyl)-isoxazolin-5-yl}-acetyl]-N²-(2-chlorophenylsulfonyl)-2,3-diaminopropanoic acid;

N³-[2-{3-(4-formamidinophenyl)-isoxazolin-5-yl}-acetyl]-N²-(4-trifluoromethylphenylsulfonyl)-2,3-diaminopropanoic acid;

N³-[2-{3-(4-formamidinophenyl)-isoxazolin-5-yl}-acetyl]-N²-(2-trifluoromethylphenylsulfonyl)-2,3-diaminopropanoic acid;

N³-[2-{3-(4-formamidinophenyl)-isoxazolin-5-yl}-acetyl]-N²-(2-fluorophenylsulfonyl)-2,3-diaminopropanoic acid;

N³-[2-{3-(4-formamidinophenyl)-isoxazolin-5-yl}-acetyl]-N²-(4-fluorophenylsulfonyl)-2,3-diaminopropanoic acid;

N³-[2-{3-(4-formamidinophenyl)-isoxazolin-5-yl}-acetyl]-N²-(4-methoxyphenylsulfonyl)-2,3-diaminopropanoic acid;

N³-[2-{3-(4-formamidinophenyl)-isoxazolin-5-yl}-acetyl]-N²-(2,3,5,6-tetramethylphenylsulfonyl)-2,3-diaminopropanoic acid;

N³-[2-{3-(4-formamidinophenyl)-isoxazolin-5-yl}-acetyl]-N²-(4-cyanophenylsulfonyl)-2,3-diaminopropanoic acid;

N³-[2-{3-(4-formamidinophenyl)-isoxazolin-5-yl}-acetyl]-N²-(4-chlorophenylsulfonyl)-2,3-diaminopropanoic acid;

N³-[2-{3-(4-formamidinophenyl)-isoxazolin-5-yl}-acetyl]-N²-(4-propylphenylsulfonyl)-2,3-diaminopropanoic acid;

N³-[2-{3-(4-formamidinophenyl)-isoxazolin-5-yl}-acetyl]-N²-(2-phenylethylsulfonyl)-2,3-diaminopropanoic acid;

N³-[2-{3-(4-formamidinophenyl)-isoxazolin-5-yl}-acetyl]-N²-(4-isopropylphenylsulfonyl)-2,3-diaminopropanoic acid;

N³-[2-{3-(4-formamidinophenyl)-isoxazolin-5-yl}-acetyl]-N²-(3-phenylpropylsulfonyl)-2,3-diaminopropanoic acid;

N³-[2-{3-(4-formamidinophenyl)-isoxazolin-5-yl}-acetyl]-N²-(3-pyridylsulfonyl)-2,3-diaminopropanoic acid;

N³-[2-{3-(4-formamidinophenyl)-isoxazolin-5-yl}-acetyl]-N²-(phenylaminosulfonyl)-2,3-diaminopropanoic acid;

N³-[2-{3-(4-formamidinophenyl)-isoxazolin-5-yl}-acetyl]-N²-(benzylaminosulfonyl)-2,3-diaminopropanoic acid;

N³-[2-{3-(4-formamidinophenyl)-isoxazolin-5-yl}-acetyl]-N²-(dimethylaminosulfonyl)-2,3-diaminopropanoic acid;

N³-[2-{3-(2-fluoro-4-formamidinophenyl)-isoxazolin-5-yl}-acetyl]-N2-(3-methylphenylsulfonyl)-2,3-diaminopropanoic acid;

N³-[2-{3-(2-formamidino-5-pyridinyl)-isoxazolin-5-yl}-acetyl]-N²-(n-butyloxycarbonyl)-2,3-diaminopropanoic acid;

N³-[2-{3-(2-formamidino-5-pyridinyl)-isoxazolin-5-yl}-acetyl]-N²-(3-methylphenylsulfonyl)-2,3-diaminopropanoic acid;

N³-[2-{3-(3-formamidino-6-pyridinyl)-isoxazolin-5-yl}-acetyl]-N2-(n-butyloxycarbonyl)-2,3-diaminopropanoic acid,

N³-[2-{3-(3-formamidino-6-pyridinyl)-isoxazolin-5-yl}-acetyl]-N²-(3-methylphenylsulfonyl)-2,3-diaminopropanoic acid,

N³-[2-{3-(4-formamidinophenyl)-isoxazolin-5-yl}-acetyl]-N²-(phenylaminocarbonyl)-2,3-diaminopropanoic acid;

N³-[2-{3-(4-formamidinophenyl)-isoxazolin-5-yl}-acetyl]-N²-(4-fluorophenylaminocarbonyl)-2,3-diaminopropanoic acid;

N³-[2-{3-(4-formamidinophenyl)-isoxazolin-5-yl}-acetyl]-N²-(1-naphthylaminocarbonyl)-2,3-diaminopropanoic acid;

N³-[2-{3-(4-formamidinophenyl)-isoxazolin-5-yl}-acetyl]-N²-(benzylaminocarbonyl)-2,3-diaminopropanoic acid;

N³-[2-{3-(4-formamidinophenyl)-isoxazolin-5-yl}-acetyl]-N²-(3-bromo-2-thienylsulfonyl)-2,3-diaminopropanoic acid;

N³-[2-{3-(4-formamidinophenyl)-isoxazolin-5-yl}-acetyl]-N²-(3-methyl-2-benzothienylsulfonyl)-2,3-diaminopropanoic acid,

N³-[2-{3-(4-formamidinophenyl)-isoxazolin-5-yl}-acetyl]-N²-(isobutyloxycarbonyl)-2,3-diaminopropanoic acid,

N³-[2-{3-(4-formamidinophenyl)-isoxazolin-5-yl}-acetyl]-N²-(isobutyloxycarbonyl)-2,3-diaminopropanoic acid,

N³-[2-{3-(4-formamidinophenyl)-isoxazolin-5-yl}-acetyl]-N²-(isobutyloxycarbonyl)-2,3-diaminopropanoic acid,

N³-[2-{3-(4-formamidinophenyl)-isoxazolin-5-yl}-acetyl]-N²-(2-cyclopropylethoxycarbonyl)-2,3-diaminopropanoic acid,

N³-[2-{3-(4-guanidinophenyl)-isoxazolin-5-yl}-acetyl]-N²-(n-butyloxycarbonyl)-2,3-diaminopropanoic acid;

N³-[2-{3-(4-guanidinophenyl)-isoxazolin-5-yl}-acetyl]-N²-(3-methylphenylsulfonyl)-2,3-diaminopropanoic acid;

N³-[2-{5-(4-formamidinophenyl)-isoxazolin-3-yl}-acetyl]-N²-(n-butyloxycarbonyl)-2,3-diaminopropanoic acid;

N³-[2-{3-(4-formamidinophenyl)-isoxazolin-5-yl}-acetyl]-N²-(2-bromo-phenylsulfonyl)-2,3-diaminopropionic acid;

N³-[2-{3-(4-formamidinophenyl)-isoxazolin-5-yl}-acetyl]-N²-(2-methyl-phenylsulfonyl)-2,3-diaminopropionic acid;

N³-[2-{3-(3-formamidino-6-pyridinyl)-isoxazolin-5-yl}-acetyl]-N²-(3-methylphenylsulfonyl)-2,3-diaminopropionic acid;

N³-[2-{3-(2-formamidino-5-pyridinyl)-isoxazolin-5-yl}-acetyl]-N²-(3-methylphenylsulfonyl)-2,3-diaminopropionic acid;

N³-[2-{3-(2-fluoro-4-formamidinophenyl)-isoxazolin-5-yl}-acetyl]-N²-(3-methylphenylsulfonyl)-2,3-diaminopropionic acid;

N³-[2-{3-(4-formamidinophenyl)-isoxazolin-5-yl}-acetyl]-N²-(3-bromo-phenylsulfonyl)-2,3-diaminopropionic acid;

N³-[2-{3-(4-formamidinophenyl)-isoxazolin-5-yl}-acetyl]-N²-(4-bromo-phenylsulfonyl)-2,3-diaminopropionic acid;

or a propionate ester prodrug form of said compound, wherein the hydrogen of the hydroxy group of the propanoic acid moiety is substituted with a group selected from:

methyl;

ethyl;

isopropyl;

methylcarbonyloxymethyl-;

ethylcarbonyloxymethyl-;

t-butylcarbonyloxymethyl-;

cyclohexylcarbonyloxymethyl-;

1-(methylcarbonyloxy)ethyl-;

1-(ethylcarbonyloxy)ethyl-;

1-(t-butylcarbonyloxy)ethyl-;

1-(cyclohexylcarbonyloxy)ethyl-;

i-propyloxycarbonyloxymethyl-;

cyclohexylcarbonyloxymethyl-;

t-butyloxycarbonyloxymethyl-;

1-(i-propyloxycarbonyloxy)ethyl-;

1-(cyclohexyloxycarbonyloxy)ethyl-;

1-(t-butyloxycarbonyloxy)ethyl-;

dimethylaminoethyl-;

diethylaminoethyl-;

(5-methyl-1,3-dioxacyclopenten-2-on-4-yl)methyl-;

(5-(t-butyl)-1,3-dioxacyclopenten-2-on-4-yl)methyl-;

(1,3-dioxa-5-phenyl-cyclopenten-2-on-4-yl)methyl-;

1-(2-(2-methoxypropyl)carbonyloxy)ethyl-.

Preferred GPIIb/IIIa antagonists useful in assays of the present invention are Compounds A, B, C and D listed below, and salt forms, prodrug forms and metabolites thereof.

Compound A referred to herein is 2(S)-[(n-butoxycarbonyl)amino]-3-[[[3-[4-(aminoiminomethyl)phenyl]isoxazolin-5(R)-yl]methylcarbonyl]amino]propionic acid or its methyl ester. The preparation of Compound A is disclosed in PCT Patent Application Publication Number WO 95/14683, incorporated herein by reference.

Compound B referred to herein is 2(S)-[[(3,5-dimethylisoxazol-4-yl)sulfonyl]amino]-3-[[[3-[4-(aminoimino methly)phenyl]isoxazolin-5(R)-yl]methyl carbonyl]amino]propionic acid. The preparation of Compound B is disclosed in PCT Patent Application Publication Number WO 96/37482, published Nov. 28, 1996, incorporated herein by reference.

Compound C referred to herein is to 2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid. The preparation of Compound C is disclosed in PCT Patent Application Publication Number WO 94/18981, incorporated herein by reference.

Compound D referred to herein is 5-[2-(piperdin-4-yl)ethyl]thieno[2,3-b]thiophene-2-N-(3-2(S)-(3-pyridinyl sulfonylamino)propionic acid]carboxamide. The preparation of Compound D is disclosed in PCT Patent Application Publication Number WO 95/14351, incorporated herein by reference.

The invention can be further understood by the following examples in which parts and percentages are by weight unless otherwise indicated. Preferred embodiments of the invention have been chosen for purposes of illustration and description, but are not intended in any way to restrict the scope of the invention. The preferred embodiments of certain aspects of the invention are shown in the accompanying drawings.

### EXAMPLE 1

Detection of GPIIb/IIIa Drug-Dependent Activating Substances (DDPASs) in a Patient Plasma Sample using the Drug-Dependent Activating Substance Flow Cytometric Assay (DDPASFCA) Experiments were performed using the DDPASFCA for the detection of platelet CD62 as follows:

50 μL of citrated plasma from a patient eliciting a thrombocytopenic response during treatment with Compound A was added to Costar Serocluster® 96 well V-bottom microtiter plates (#3897). GPIIb/IIIa antagonist or vehicle was added (2 μL of 5μM compound A, final concentration 200 nM) was added, followed by sufficient PRP or PRP diluted into PPP to give a final platelet number of 1×10⁶/well (typically 2 μL PRP). Reactions were incubated without shaking for 120 minutes. After this time, 15 μL of phycoerythrin conjugated anti-CD62 (anti-CD62-PE, Bectin Dickinson) was added. After 30 minutes the samples were diluted with 130 μL flow buffer (FB) consisting of 10 mM HEPES, 5 mM Kcl, 168 mM NaCl, 1 mM MgCl₂. Samples were then transferred to 12×75 mm polystyrene and analyzed on a FACScan (Bectin Dickinson). PE fluorescence was read on FL2. Platelets were identified by their characteristic forward and side light scatter. Data from 10,000 events was obtained per sample and analyzed using Bectin Dickinson CellQuest software. The effects of GPIIb/IIIa antagonists on CD62 expression are expressed as the difference in median PE fluorescence for reactions in the presence of GPIIb/IIIa antagonist and CD62 median PE fluorescence in the absence of a GPIIb/IIIa antagonist.

### EXAMPLE 2

Modifications to the DDPASFCA to Detect DDPASs in Plasma Samples using the Donor's Own (Versus Heterologous) Platelets

The occurrence of DDPASs in the general population was tested by a modification of the DDPASFCA using the donor's own platelets to conduct the assay. PRP (100 μL) from healthy human donors was treated with Compound A (200 nM) or vehicle for 90 minutes. A 5μL sample was transferred to microtiter wells containing 20 μL anti-CD62-PE and samples analyzed by flow cytometry as described in Example 1. Positive samples (defined as Delta PE fluorescence of >0) were retested using the method of Example 1 (heterologous donor). 8% of donors were weakly positive (delta between 10 and 25). One sample (2%) was significantly positive (delta 566). Thus, the prevalence and titer of pre-existing DDPASs is relatively low in the general population. These results indicate that pre-existing DDPASs can be detected by the GPIIb/IIIa DDPASFCA of the present invention.

### EXAMPLE 3

Detection of GPIIb/IIIa DDPASs in Patients Subsequently Treated with Compound A

Citrated pre-dose plasma samples from patients subsequently dosed with Compound A who did not develop a clinically significant thrombocytopenic response were analyzed for the presence of pre-existing DDPASs using the DDPASFCA of the present invention (using Compound A as the GPIIb/IIIa antagonist in the assay).

The procedure was the same as in Example 1 (with modifications outlined in that example) except that where samples were limiting, only 35-40 μL of plasma was used. The occurrence of pre-existing titers was found to be low in this group of patients dosed with Compound A (2/78, 2.5%), suggesting that the assays of the present invention will have predictive value for determining the risk of the occurrence of thrombocytopenic/thromboembolic episode mediated by DDPASs associated with GPIIb/IIIa antagonist treatment.

Citrated plasma samples from patients subsequently dosed with Compound A who did not develop a clinically significant thrombocytopenic/thromboembolic responses, but were positive for the DDAB ELISA, were analyzed for the presence of pre-existing (pre-dose) and subsequent (study exit) DDPASs using the DDPASFCA of the present invention and the procedure of Example 1 (using Compound A as the GPIIb/IIIa antagonist in the assay). There were no substantial DDPASs in these plasmas either before or after subsequent treatment with Compound A except for sample 099016. This data indicates that some, but not all DDAB positive samples have drug-dependent platelet activating activity.

### EXAMPLE 4

Specificity of the GPIIb/IIIa DDPASFCA Indicating the Assay Detects DDPASs that are not Immunoglobulins

Plasma from a patient who developed a thrombocytopenic episode while under therapy with Compound A was analyzed for the presence of DDPASs that might not be of immunoglobulin nature. Plasma from this previously thrombocytopenic patient (taken 17 months after the thrombocytopenia) as well as normal human plasma (negative control) and plasma from a subject known to contain DDPAS (positive control) were processed to remove immunoglobulins by passage through 1 mL protein A Hitrap® columns (Pharmacia, Inc.). The immunoglobulin-depleted plasma was free of IgG as assessed by an IgGl-specific ELISA and negative for the presence of GPIIb/IIIa antagonist-dependent anti-platelet antibodies as assessed by the DDAB ELISA assay. Plasma samples were then tested in the DDPASFCA as described in Example 1. There was no statistical difference between results for plasma samples containing immunoglobulin and those not containing immunoglobulin, Table 1.

TABLE 1

| | FL2 | FL2 | | IgG | DDAB |
|---|---|---|---|---|---|
| Plasma | no comp'd | compound A | Delta | status | ELISA |
| TCP | 76 +/− 2 | 154 +/− 8 | 78 +/− 10 | + | + |
| TCP | 62 +/− 5 | 156 +/− 20 | 94 +/− 25 | − | − |
| Neg. | 18 +/− 1 | 16 +/− 1 | −2 +/− 2 | + | − |
| control | | | | | |
| Neg. | 44 +/− 6 | 30 +/− 9 | −44 +/− 15 | − | − |
| control | | | | | |
| Pos. | 173 +/− 46 | 315 +/− 27 | 142 +/− 73 | + | + |
| control | | | | | |
| Pos. | 175 +/− 19 | 297 +/− 33 | 122 +/− 52 | − | − |
| control | | | | | |

FL2=Median fluorescence read on flow cytometer for analysis of phycoerythrin (PE); Delta=difference in median PE fluorescence in the presence of compound A and CD62 median PE fluorescence in the absence of compound A; IgG status: +=plasma, −=protein A-depleted plasma; TCP=plasma from patient who previously had a thrombocytopenic response to compound A. Pos. control=individual with DDPASFCA positive plasma.

### EXAMPLE 5

Use of the GPIIb/IIIa DDPASFCA to Detect Platelet Activating Drug Dependent Antibodies (PADDABs)

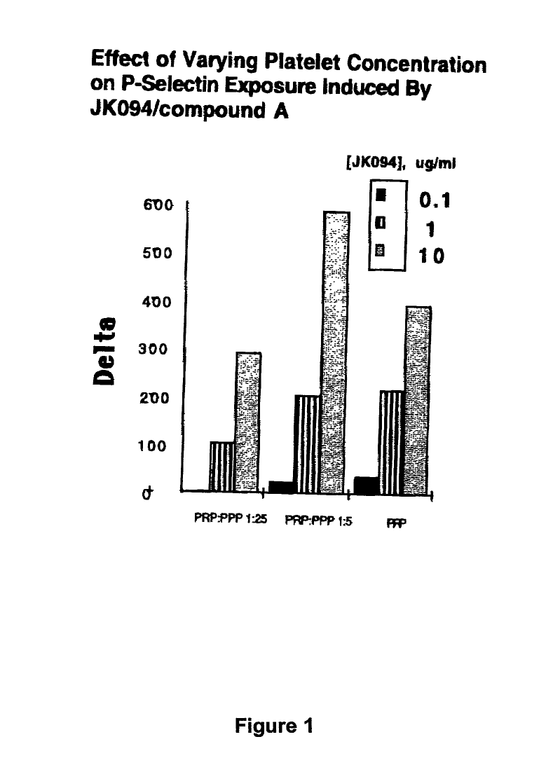

Monoclonal antibody JK094 binds to human platelets in the presence of many GPIIb/IIIa antagonists, such as Compound A. The ability of the DDPASFCA to detect a drug-dependent activating effect of this binding was monitored by incubating platelets (at increasing concentrations) with 3 concentrations of JK094 in the presence of 1000 nM compound A. After 70 minutes an aliquot of the reaction containing ˜1×10⁶ platelets was transferred to microtiter wells containing 20 μL of anti-CD62-PE, and analyzed by flow cytometry as in Example 1. The data show that JK094 is a PADDAB. (FIG. 1)

### EXAMPLE 6

Detection of ADP as a GPIIb/IIIa DDPAS by DDPASFCA

Adenosine diphosphate (ADP) was evaluated as a possible DDPAS as follows: Compound A (final concentration 1 μM) or vehicle was added to 25 μL of freshly prepared PRP in wells of a 96-well microtiter plate. One set of wells received 3 μL of ADP at the final concentration indicated in Table 2. Another set of wells received vehicle. After 10 minutes, 2.5 μL of from each well was transferred to microtiter wells containing only 20 μL of anti-CD62-PE. After incubation for 30 minutes, samples were analyzed by flow cytometry as in Example 1.

TABLE 2

| | | no | | | | |
|---|---|---|---|---|---|---|
| | no | compound | | +compound | | Delta |
| [ADP], | compound | Stdev | + compound | Stdev | Delta | Stdev |
| (final) μM | Med F12 | Med F12 | Med F12 | Med F12 | Med F12 | Med F12 |
| 10 | 126 | 6 | 208 | 16 | 82 | 22 |
| 2 | 48 | 3 | 89 | 5 | 40 | 9 |
| 0.4 | 14 | 1 | 20 | 6 | 5 | 7 |
| 0 | 12 | 0 | 11 | 0 | −1 | 0 |

### EXAMPLE 7

Determination of a DDPAS by the DDPASFCA as a Function of the Concentration of the DDPAS

The detection of a positive DDPAS titer in the presence of a donor plasma was assessed with dilutions of the DDPASFCA-positive plasma DPC38 into the DDPASFCA-negative plasma DPC50 using platelets from DPC50 as follows: Varying volume amounts of control plasma (DPC50), not containing DDPASs, were mixed with a the DDPAS-positive test plasma (total volume 50 μL). 1×10⁶ platelets (PRP) was added. Samples were treated as in Example 1 with and without added Compound A. In the concentration range employed, DDPASs could be quantitatively measured in the test plasma. This result indicates that plasma constituents do not interfere with the detection of low titer DDPASs (Table 3). Thus, the signal intensity in the DDPASFCA was dose-dependent with respect to the plasma concentration for the DDPASFCA-positive subject DPC38. In contrast DDPASs were undetectable in the control plasma.

TABLE 3

| DPC38 | DPC50 | | | |
|---|---|---|---|---|
| volume | volume | No compound | + compound | Delta |
| (ul) | (ul) | Med F12 | Med F12 | Med F12 |
| 50 | 0 | 366 | 670 | 304 |
| 25 | 25 | 192 | 271 | 79 |
| 17 | 33 | 114 | 135 | 21 |
| 12 | 38 | 71 | 88 | 17 |
| 10 | 40 | 57 | 73 | 17 |
| 8 | 42 | 44 | 64 | 20 |
| 0 | 50 | 20 | 12 | −8 |

### EXAMPLE 8

Use of the DDPASFCA to Detect Changes in DDPAS Titer Over Time

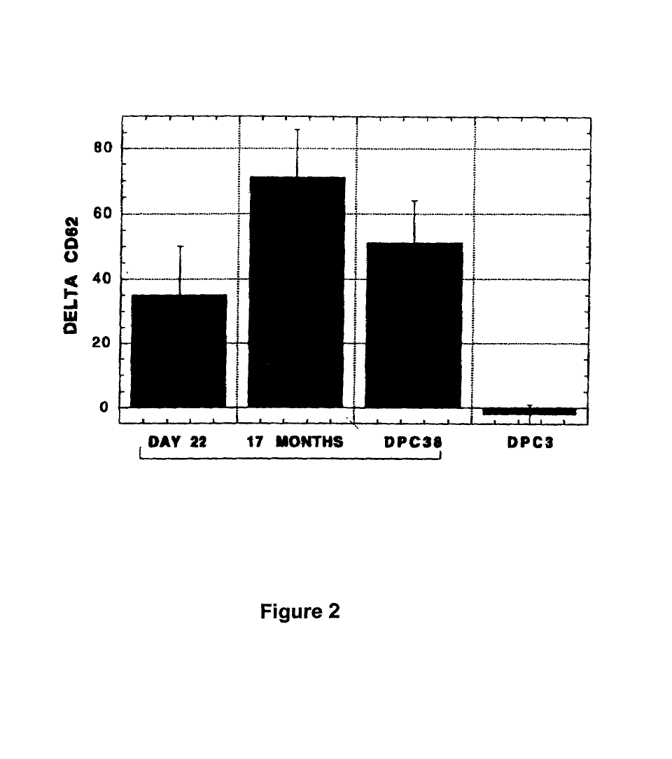

The development of DDPASs in the plasma of a patient who received Compound A and developed thrombocytopenia was analyzed using the procedure of Example 1. DDPASFCA was not determined for patient #304 prior to taking Compound A. The patient experienced thrombocytopenia after 3 days of administration of Compound A. DDPASs were subsequently detected in this patient's plasma on day 22 post-administration of Compound A and were also detected 17 months later (FIG. 2). DPC38 was used as a positive control. DPC3 was used as a negative control. The detection of a DDPAS titer in this patient suggests that the assay of the present invention may be used to monitor patients before, during and after GPIIb/IIIa antagonist treatment to identify patients with DDPASs or increasing DDPASs who may be at risk of developing thrombocytopenia. In such patients the GPIIb/IIIa antagonist treatment may not be started, or may be terminated or treatment may be switched to a GPIIb/IIIa antagonist that does not potentiate the activity of platelet activating substances present in the patient's blood. In a prospective study, a patient with such pre-existing DDPASs could be excluded from the study, possibly preventing the clinically significant thrombocytopenic episode.

### EXAMPLE 9

Certain DDPASs can be Removed from a Sample by Platelets Treated with GPIIb/IIIa-Antagonists

Thrombocytopenic patient 099016 plasma was evaluated in the DDPASFCA as in Example 1, with the modification that 10 μL of sample, or a sample of 099016 plasma diluted into DDPASFCA-negative plasma, was combined with 20 μL donor PRP (final platelet concentration=1×10⁸/mL). The signal in the DDPASFCA was proportional to the patient plasma dilution. Next, the ability of platelets, a physiologically relevant source of GPIIb/IIIa, to remove certain kinds of DDPASs was tested. DDPAS positive thrombocytopenic plasma 099016 (70 μl) was treated with fresh donor PRP (140 μL, final platelet concentration 3×10⁸/mL) for 60 minutes in the presence or the absence of compound A (1 μM). Platelets were removed by centrifugation at 1000×G for 5 minutes, and the resulting plasma samples were tested in the DDPASFCA as in Example 1, with the modification that 3 μL of sample was combined with 27 μL donor PRP (final platelet concentration=1×10⁸/mL) in the presence or the absence of Compound A (1 μM). Only plasma exposed to both platelets and Compound A were depleted of the DDPAS. In a related experiment, the platelet pellets resulting from depletion with platelets in the presence and the absence of Compound A were resuspended in 70 μL of 9 mM EDTA and heated at 37° C. for 2 hours. After this time the supernatant was recovered after centrifugation at 1700×G for 30 minutes at 4° C. and the [CaCl₂]was adjusted to 15 mM. A potent thrombin inhibitor was added to a final concentration of 5μM. The samples were then evaluated in the DDPASFCA. Compared to the negative controls DPC43 and DPC44, only the sample recovered from platelet+Compound A depletion of 099016 plasma was substantially positive. These data indicate that certain DDPASs are capable of binding to, and being removed by, platelets in a GPIIb/IIIa-antagonist dependent manner.

### EXAMPLE 10

Specific Distribution and Recovery of Thrombocytopenic Patient 099016 DDABs onto Platelets by Compound A

Thrombocytopenic patient 099016 plasma was processed with platelets in the presence or the absence of Compound A to deplete any DDAB as described in Example 9. After treatment of 099016 plasma with platelets in the presence and in the absence of Compound A, samples were evaluated in the DDAB ELISA at 3 dilutions (1/100, 1/250 and 1/500) for residual DDAB. Murine JK094 was used as a positive control for the ELISA. Treatment of 099016 plasma with donor platelets resulted in no loss of detectable DDAB, whereas treatment with donor platelets in the presence of compound A specifically depleted the DDAB. This shows the drug-specific nature of this anti-platelet antibody. ELISA analysis of the EDTA elutants from platelets treated with 099016 plasma without Compound A were devoid of DDAB, while EDTA eluants from platelets treated with 099016 plasma with Compound A showed DDAB.

### EXAMPLE 11

Use of Alternative Microtiterplates to Increase the Sensitivity of the DDPASFCA

ADP and DPC38 were used as sources of DDPASs. 2 μL of citrated PRP (DPC3) was added to either Costar Serocluster® 96 well V-bottom microtiterplates (#3897) or #3898 microtiter plates (Costar Serocluster®) (rated as “hydrophilic” in nature by the manufacturer). Platelets were then treated with and without Compound A (200 nM) with the indicated concentrations of ADP. Similarly, DDPAS positive PRP (DPC38) was added to wells of both types of plates and treated with and without Compound A (200 nM). Reactions were incubated without shaking for 120 minutes. After this time, 20 μL of phycoerythrin conjugated anti-CD62 (anti-CD62-PE, Bectin Dickinson) was added. After 30 minutes the samples were diluted with 150 μL flow buffer and bound anti-CD62-PE measured as described in Example 1. As shown in Table 4, there is an improvement in sensitivity with Costar Serocluster® #3898 microtiter plates as compared to Costar Serocluster® #3837 microtiter plates without introducing false positives (DDPASFCA negative plasma DPC50 remains negative with #3898 microtiter plates).

TABLE 4

| Platelet | | | |
|---|---|---|---|
| donor | [ADP], uM | Delta CS | Delta H |
| DPC3 | 0 | 24 | 28 |
| DPC3 | 10 | 89 | 157 |
| DPC3 | 100 | 82 | 168 |
| DPC38 | 0 | 190 | 338 |
| DPC50 | 0 | 0 | −1 |

Delta SC=Delta Fluorescence for Costar Serocluster microtiter plates

Delta H=Delta Fluorescence for H3898 microtiter plates

What is claimed is:

1. A method for detecting drug-dependent platelet activating substances in a subject which recognize an integrin bound with an integrin antagonist/agonist comprising:
(a) incubating platelets with one or more selected integrin antagonists/agonists, to form a complex between integrin and the selected integrin antagonist/agonist;
(b) incubating the platelet:integrin antagonist/agonist mixture of step (a) with a sample containing a DDPAS from the subject;
(c) incubating the platelet:integrin antagonist/agonist mixture of step (b) with a labeled secondary anti-human CD62 antibody, to form a complex between the labeled secondary anti-human CD62 and CD62 on the platelet surface; and
(d) detecting the labeled secondary antibody.

2. A method of claim 1 wherein the integrin is GPIIb/IIIa.

3. A method of claim 1 wherein the selected integrin antagonist of step (a) is selected from one or more of the following compounds or an active metabolite form thereof:
2(S)-[(n-butoxycarbonyl)amino]-3-[[[3-[4-(aminoiminomethyl)phenyl]isoxazolin-5(R)-yl]methylcarbonyl]amino]propionic acid;
2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;
2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid; and
5-[2-(piperdin-4-yl)ethyl]thieno[2,3-b]thiophene-2-N-(3-2(S)-(3-pyridinylsulfonylamino)propionic acid]carboxamide.

4. A method of claim 1 wherein the labeled secondary anti-human antibody is an anti-human CD62 antibody conjugated with an enzyme or an anti-human CD62 antibody conjugated with a fluorescent label.

5. A method of claim 4 wherein the enzyme is horseradish peroxidase.

6. A method of claim 4 wherein the fluorescent label is phycoerythrin or fluorescein or a derivative thereof.

7. A method of claim 1 wherein the sample containing a DDPAS is plasma obtained from the subject.

8. A method for identifying a subject having risk of developing thrombocytopenia/thromboembolic complications during treatment with an integrin antagonist/agonist, wherein platelets are selected from a platelet rich plasma (PRP) from the subject, PRP from the subject diluted with plasma from the subject, or PRP from a healthy human donor diluted with plasma from the subject, comprising:
(a) incubating platelets with one or more selected integrin antagonists/agonists to form a complex between integrin and the selected integrin antagonist/agonist;
(b) incubating the platelet:integrin antagonist/agonist mixture of step (a) with a labeled secondary anti-human CD62 antibody, to form a complex between the labeled secondary anti-human CD62 antibody and CD62 on the platelet surface;
(c) measuring the amount of formation of the complex between the labeled secondary anti-human CD62 antibody and CD62 on the platelet surface of step (b), by detection of the labeled secondary anti-human CD62 antibody label; and
(d) comparing the amount of formation of the complex between the labeled secondary anti-human CD62 antibody and CD62 on the platelet surface of step (c) with the amount of such complex formed when steps (b), (c), and (d) are carried out and step (a) is omitted.

9. A method of claim 8 wherein the sample containing DDPAS is obtained from the subject and the method is performed prior to treatment of the subject with an integrin antagonist/agonist.

10. A method of claim 8 wherein the sample containing DDPAS is obtained from the subject and the method is performed concurrently with treatment of the subject with an integrin antagonist/agonist.

11. A method of claim 8 wherein the selected integrin antagonists/agonists of step (a) comprise the active form or active metabolite of the integrin antagonist/agonist which is used to treat the subject.

12. A method of claim 8 wherein the selected integrin antagonist of step (a) is selected from one or more of the following compounds or an active metabolite form thereof:
2(S)-[(n-butoxycarbonyl)amino]-3-[[[3-[4-(aminoiminomethyl)phenyl]isoxazolin-5(R)-yl]methylcarbonyl]amino]propionic acid;
2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;
2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid; and
5-[2-(piperdin-4-yl)ethyl]thieno[2,3-b]thiophene-2-N-(3-2(S)-(3-pyridinylsulfonylamino)propionic acid]carboxamide.

13. A method of treating a subject with an integrin antagonist/agonist, comprising:
(a) performing the method of claim 8 wherein the sample containing DDPAS is obtained from the subject and the method is performed prior to treating the subject with the integrin antagonist/agonist;
(b) administering to the subject an effective amount of a pharmaceutical composition comprising the integrin antagonist/agonist; and
(c) performing the method of claim 8 wherein the sample containing DDPAS is obtained from the subject and the method is performed concurrently with treatment of the subject with the integrin antagonist/agonist.

14. A method of claim 13 wherein the subject is treated with an integrin antagonist selected from one or more of the following compounds:
2(S)-[(n-butoxycarbonyl)amino]-3-[[[3-[4-(aminoiminomethyl)phenyl]isoxazolin-5(R)-yl]methylcarbonyl]amino]propionic acid or the methyl ester thereof;
2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;
2(S)-[(4-methylphenylsulfonyl)amino]-3-[[[5,6,7,8-tetrahydro-4-oxo-5-[2-(piperidin-4-yl)ethyl]-4H-pyrazolo-[1,5-a][1,4]diazepin-2-yl]carbonyl]amino]propionic acid; and
5-[2-(piperdin-4-yl)ethyl]thieno[2,3-b]thiophene-2-N-(3-2(S)-(3-pyridinylsulfonylamino)propionic acid]carboxamide.

15. A diagnostic flow cytometry kit, comprising: at least one selected integrin antagonist/agonist and a secondary labeled anti-human CD62 antibody to be used in conjunction with a source of platelets.

16. A method of determining whether a selected integrin antagonist/agonist potentiates the exposure of CD62 in a subject who's blood recognizes an integrin bound with an integrin antagonist/agonist, comprising:
(a) incubating platelets with one or more selected integrin antagonists/agonists to form a complex between integrin and the selected integrin antagonist/agonist;
(b) incubating the platelet:integrin antagonist/agonist mixture of step (a) with a sample containing a DDPAS from the subject; and
(c) incubating the platelet:integrin antagonist/agonist mixture of step (b) with a labeled secondary anti-human CD62 antibody, to form a complex between the labeled secondary anti-human CD62 and CD62 on the platelet surface; and
(d) detecting the labeled secondary antibody.
